(19) United States
(12) Patent Application Publication
(10) Pub. No.: US 2005/0043814 A1
(43) Pub. Date: Feb. 24, 2005

(54) Acellular matrix implanted into an articular cartilage or osteochondral lesion protected with a biodegradable polymer modified to have extended polymerization time and methods for preparation and use thereof

(76) Inventors: Akihiko Kusanagi, Brookline, MA (US); Laurence J. B. Tarrant, Northampton, MA (US)

Correspondence Address: Hana Verny;Peters, Verny, Jones & Schmitt LLP, Suite 230, 425 Sherman Avenue, Palo Alto, CA, 94306, US

(21) Appl. No.: 10/921,389

(22) Filed: Aug. 18, 2004

Related U.S. Application Data
(60) Provisional application No. 60/496,971, filed on Aug. 20, 2003.

(51) Int. Cl. A61F 2/28

(52) U.S. Cl. 623/23.58; 435/397; 606/77; 623/14.12; 623/908

(57) ABSTRACT

Acellular matrix implants for implantation into an articular cartilage or osteochondral lesion and a method for fabrication thereof. A protective biodegradable polymer barrier having extended polymerization time and a method for preparation thereof. Bone-inducing compositions. A method for treatment of articular cartilage or osteochondral injuries.

### BACKGROUND OF THE INVENTION

This application is based on and claims priority of the Provisional Application Serial No.: 60/496,971, filed Aug. 20, 2003.

### FIELD OF INVENTION

The current invention concerns acellular matrix implants implanted into an articular cartilage or osteochondral lesion protected with a biodegradable polymer barrier modified to have extended polymerization time. The invention further concerns compositions for treatment of articular cartilage or osteochondral defects and injuries and a method for treatment of articular cartilage or osteochondral defects using an acellular matrix implant implanted into a joint cartilage lesion and/or into the osteochondral defect in situ wherein the osteochondral defect is further implanted with a bone-inducing composition or a carrier comprising said composition. In particular, the current invention concerns acellular matrix implants implanted into the articular cartilage or osteochondral lesion protected with a protective biodegradable polymer barrier wherein said polymer is modified to have extended polymerization time between 2 and 10 minutes, preferably between 3 to 5 minutes.

The acellular matrix implant of the invention comprises a two or three dimensional biodegradable scaffold structure implanted into the joint cartilage lesion over or below one layer, or between two layers, of biologically acceptable biodegradable polymer modified to have an extended polymerization time.

The method for treatment of articular cartilage comprises preparation of a modified protective biodegradable polymer as an insulation barrier for the acellular implant, preparation of the acellular implant, preparation of the lesion for implantation of said implant including a step of depositing the protective biodegradable polymer barrier at the bottom of the cartilage lesion for sealing the joint cartilage lesion and protecting the implant from effects of blood-borne agents, implanting the implant of the invention into the lesion and depositing a second protective biodegradable polymer barrier over the implant.

The method for treatment of osteochondral defects additionally comprises depositing a bone-inducing composition, or a carrier comprising said composition, into the bone lesion wherein said bone lesion is covered by a layer of the protective biodegradable polymer barrier thereby separating said bone and cartilage lesions from each other.

Additionally, the invention concerns methods for fabrication of an acellular implant of the invention, a bone-inducing composition or a carrier comprising said composition and for preparation of the protective biodegradable polymer having extended polymerization time between 2 and 10 minutes.

### BACKGROUND AND RELATED DISCLOSURES

Damage to the articular cartilage which occurs in active individuals and older generation adults as a result of either acute or repetitive traumatic injury or aging is quite common. Such damaged cartilage leads to pain, affects mobility and results in debilitating disability.

Typical treatment choices, depending on lesion and symptom severity, are rest and other conservative treatments, minor arthroscopic surgery to clean up and smooth the surface of the damaged cartilage area, and other surgical procedures such as microfracture, drilling, and abrasion. All of these may provide symptomatic relief, but the benefit is usually only temporary, especially if the person's pre-injury activity level is maintained. For example, severe and chronic forms of knee joint cartilage damage can lead to greater deterioration of the joint cartilage and may eventually lead to a total knee joint replacement. Nowadays, approximately 200,000 total knee replacement operations are performed annually. The artificial joint generally lasts only 10 to 15 years and the operation is, therefore, typically not recommended for people under the age of fifty.

Osteochondral diseases or injuries, which are combination lesions of bone and cartilage, present yet another challenge for a treatment of which need is not being met by the currently available procedures and methods. For example, treatment of osteochondritis dissecans with autologous chondrocyte transplantation, described in J. Bone and Joint Surgery, 85A-Supplement 2: 17-24 (2003), requires multiple surgeries and at least three weeks for cell cultivation and growth.

It would, therefore, be extremely advantageous to have available a method for in situ treatment of these injuries which would effectively restore the cartilage or bone to its pre-injury state during one surgery and with minimal time needed for recovery, which treatment would be especially suitable for younger individuals who are more active and have better recovery capabilities.

Attempts to provide means and methods for repair of articular cartilage are disclosed, for example, in U.S. Pat. Nos. 5,723,331; 5,786,217; 6,150,163; 6,294,202; 6,322,563 and in the U.S. patent application Ser. No. 09/896,912, filed on Jun. 29, 2001.

U.S. Pat. No. 5,723,331 describes methods and compositions for preparation of synthetic cartilage for the repair of articular cartilage using ex vivo proliferated denuded chondrogenic cells seeded ex vivo, in the wells containing adhesive surface. These cells redifferentiate and begin to secrete cartilage-specific extracellular matrix thereby providing an unlimited amount of synthetic cartilage for surgical delivery to a site of the articular defect.

U.S. Pat. No. 5,786,217 describes methods for preparing a multi-cell layered synthetic cartilage patch prepared essentially by the same method as described in '331 patent except that the denuded cells are non-differentiated, and culturing these cells for a time necessary for these cells to differentiate and form a multicell layered synthetic cartilage.

U.S. application Ser. No. 09/896,912, filed on Jun. 29, 2001 concerns a method for repairing cartilage, meniscus, ligament, tendon, bone, skin, cornea, periodontal tissues, abscesses, resected tumors and ulcers by introducing into tissue a temperature dependent polymer gel in conjunction with at least one blood component which adheres to the tissue and promotes support for cell proliferation for repairing the tissue.

U.S. patent applications Ser. Nos: 10/104,677; 10/625,822; 10/625,245 and 10/626,459 filed on Jul. 22, 2003, by inventors, hereby incorporated by reference, disclose neo-cartilage constructs subjected to an algorithm of certain specific conditions suitable for repair of injured or damaged articular cartilage.

None of the above cited references, however, results in repair and regeneration of cartilage or bone in situ without a need for several surgeries.

Rapid-gelling polymeric compositions for a rapid formation of a gel in situ having a polymerization time lower than one minute for adhesion to the tissue useful as tissue sealants are disclosed in U.S. Pat. Nos. 6,312,725B1 and 6,624,245 B2 and in J. Biomed. Mater. Res., 58:545-555 (2001) and the American Surgeon, 68:553-562 (2002). These sealants have very rapid polymerization time of several seconds to less than one minute which is not practicable for use as the insulation barrier for the purposes of the current invention where the surgeon requires at least two minutes but prefers to have at least three to five minutes time to deposit the insulation barrier at the bottom of the cartilage or osteochondral lesion and to be able to apply the polymers evenly over the bottom of the lesion and over the implant.

It is thus a primary objective of this invention to provide a method and a means for treatment of injured or traumatized cartilage or cartilage-bone defects by providing an acellular matrix implant, a bone-inducing composition or a carrier comprising a bone-inducing composition, and a protective biodegradable polymer having an extended polymerization time and implanting said acellular implant into said cavity between two layers of the protective biodegradable polymers. The method performed according to the invention results in formation and restoration of a healthy hyaline articular cartilage.

All patents, patent applications and publications cited herein are hereby incorporated by reference.

### SUMMARY

One aspect of the current invention is an acellular matrix implant for treatment of defects and injuries of articular cartilage.

Another aspect of the current invention is an acellular matrix implant in combination with a bone-inducing composition or a carrier comprising said composition for treatment of osteochondral defects and injuries.

Yet another aspect of the current invention is a method for fabrication of an acellular matrix implant of the invention.

Still another aspect of the current invention is a method for preparation of an acellular matrix implant wherein said matrix is a sponge, honeycomb, scaffold, thermo-reversible gelation hydrogel (TRGH), polymer of an aromatic organic acid or an absorbable caprolactone polymer.

Yet another aspect of the current invention is a method for treatment of injured, damaged, diseased or aged articular cartilage using the acellular matrix implant implanted into a joint cartilage lesion in situ.

Still yet another aspect of the current invention is a method for treatment of osteochondral defects by implanting an acellular matrix implant into the cartilage lesion in conjunction with depositing a bone-inducing composition or a carrier comprising said composition into an osteochondral lesion in situ.

Still another aspect of the current invention is a bone-inducing composition or a carrier comprising said composition containing bone-inducing agents such as a demineralized bone powder, calcium phosphate, hydroxyapatite, organoapatite, titanium oxide, poly-L-lactic or polyglycolic acid or a copolymer thereof or a bone morphogenic protein used in a method wherein said composition is deposited into the bone lesion of the osteochondral defect.

Still yet another aspect of the current invention is a bone-inducing composition or a carrier comprising said composition deposited into a bone lesion of the osteochondral defect in conjunction with implantation of an acellular matrix implant into the cartilage lesion useful for treatment of osteochondral defects.

Still yet another aspect of the current invention is a bone-inducing composition or a carrier comprising said composition deposited into a bone lesion for treatment of a bone defect in conjunction with implantation of an acellular matrix implant into the cartilage lesion or osteochondral implant useful for treatment of osteochondral defects.

Yet another aspect of the current invention is a method for treatment of injured, damaged, diseased or aged articular cartilage using an acellular matrix implant implanted into a joint cartilage lesion in situ, said method further comprising a formation of a new superficial cartilage layer overgrowing and protecting the lesion in the joint articular cartilage by applying one protective biodegradable polymer barrier at the bottom of the lesion and further applying a second protective biodegradable polymer barrier over the lesion, said bottom protective biodegradable polymer barrier providing protection of the lesion against cell and blood debris migration into the lesion from the subchondral area.

Another aspect of the current invention is a method for treatment of osteochondral defects by depositing a bone-inducing composition comprising bone-inducing agents, or a carrier comprising said composition, into a bone lesion, depositing one protective biodegradable polymer barrier over the bone-inducing composition, implanting an acellular matrix implant into the articular lesion and depositing a second protective biodegradable polymer barrier over the acellular matrix implant.

Another aspect of the current invention is a method for preparation of a protective biodegradable polymer which has no cell toxicity and is modified to have an extended polymerization time wherein said polymer comprises a linear or branched chain polyethylene glycol derivatized with tetra-succinimidyl and a linear or branched chain polyethylene glycol derivatized with tetra-thiol, in a combination, further cross-linked with alkylated collagen, the polymer modified to be substantially non-toxic to cells and tissues, have a polymerization time of at least 2 minutes and the pH adjusted to 7.5 or lower.

Still another aspect of the current invention is an acellular matrix implant for use in treatment of the cartilage or bone lesions comprising a two or three dimensional biodegradable sponge, honeycomb, hydrogel, scaffold or a polymer of an aromatic organic acid matrix implanted into the joint cartilage lesion between two layers of biologically acceptable protective biodegradable polymers.

Still yet another aspect of the current invention is a method for treatment of articular cartilage injury comprising steps:
a) preparation of an acellular matrix implant;
b) preparation of a cartilage lesion for implantation of said implant, including a step of depositing one layer of a protective biodegradable polymer barrier at the bottom of the cartilage lesion for sealing of said lesion and protecting the implant from migration of blood-borne agents;
c) implanting the implant into the lesion; and
d) depositing a second protective biodegradable polymer barrier over the acellular matrix implant,
wherein both the first and second protective biodegradable polymers have an extended polymerization time of 2 minutes or more.

Still yet another aspect of the current invention is a method for repair and restoration of damaged, injured, diseased or aged cartilage to a functional cartilage, said method comprising steps:
a) preparing an acellular matrix implant as a collagenous sponge, collagenous porous scaffold or honeycomb, thermo-reversible gelation hydrogel (TRGH), polymer of an aromatic organic acid matrix or an absorbable caprolactone polymer, wherein said sponge, scaffold, polymer of the aromatic organic acid or TRGH are biodegradable, will disintegrate with time and be metabolically removed from the healed lesion and replaced with a hyaline cartilage, said matrix optionally comprising matrix remodeling enzymes, such as matrix metalloproteinases, aggrecanases, cathepsins and/or other biologically active components;
b) introducing a layer of a protective biodegradable polymer barrier at the bottom of a cartilage lesion wherein said polymer is modified to have an extended polymerization time between 2 and 10 minutes;
c) implanting said implant into said lesion protected by the bottom layer of said protective biodegradable polymer barrier; and
d) introducing a second layer of a protective biodegradable polymer barrier over said implant wherein said protective biodegradable polymer barrier may or may not be the same as the bottom protective biodegradable polymer barrier and wherein a combination of said implant and said second protective biodegradable polymer barrier results in formation and growth of a superficial cartilage layer over the cartilage lesion in situ.

Still another aspect of the current invention is an acellular matrix implant comprising a thermo-reversible gelation hydrogel (TRGH) deposited into a lesion cavity formed above a bottom protective biodegradable polymer barrier layer and covered by the second protective biodegradable polymer layer, said TRGH deposited into said lesion either incorporated into a collagenous sponge or scaffold or as a sol at temperatures between about 5 to about 30° C., wherein within said lesion and at the body temperature said TRGH converts from the fluidic sol into a solid gel and, in this form, its presence provides a structural support for formation of extracellular matrix and generation of the hyaline cartilage, wherein said TRGH is biodegradable, will disintegrate with time and be metabolically removed from the lesion and replaced with a hyaline cartilage.

Still yet another aspect of the current invention is a method for treatment of osteochondral defects, said method comprising steps:
a) preparing a bone-inducing composition or a carrier comprising said composition comprising one or several bone-inducing agents for implantation into a bone lesion;
b) preparing an acellular matrix implant for implantation into a cartilage lesion as a collagenous sponge, collagenous porous scaffold or honeycomb or thermo-reversible gelation hydrogel (TRGH) matrix support wherein said sponge, scaffold or TRGH are biodegradable, will disintegrate with time and be metabolically removed from the lesion and replaced with a hyaline cartilage, said matrix optionally comprising matrix remodeling enzymes, matrix metalloproteinases, aggrecanases and cathepsins;
c) introducing said bone-inducing composition or a carrier comprising said composition into a bone lesion;
d) covering said bone-inducing composition or a carrier comprising said composition with one layer of a modified protective biodegradable polymer barrier;
e) implanting said acellular matrix implant into said cartilage lesion over the first layer of the protective biodegradable polymer barrier; and
f) introducing a second layer of the modified protective biodegradable polymer barrier over said implant, wherein said first and second protective biodegradable polymers may or may not be the same and wherein a combination of said acellular matrix implant and said second protective biodegradable polymer barrier results in formation and growth of a superficial cartilage layer overgrowing the cartilage lesion in situ.

### BRIEF DESCRIPTION OF DRAWINGS

FIG. 1A is an enlarged schematic representation of the cartilage lesion within the host cartilage with underlaying uninjured bone, showing a first layer of a protective biodegradable polymer barrier deposited at the bottom of the lesion, an acellular matrix implant deposited over the protective biodegradable polymer barrier and covered with a second protective biodegradable polymer barrier. FIG. 1B is an enlarged schematic representation of the osteochondral defect showing the articular lesion, bone lesion, emplacement of the bone-inducing composition (bone material) or a carrier comprising said composition into the bone lesion, emplacement of the first and second protective biodegradable polymer barriers and emplacement of the acellular matrix implant. FIG. 1C is an enlarged schematic representation of the bone defect showing the articular lesion, and combined osteochondral and skeletal bone lesion, emplacement of the bone-inducing composition or a carrier comprising said composition into the bone and osteochondral lesion, emplacement the first and second protective biodegradable polymer barriers and emplacement of the acellular matrix implant. FIG. 1D is a schematic depiction of creation of defects A and B at weight bearing site for implantation of an acellular matrix implant or serving as an empty control defect.

FIG. 2A is an image of an acellular matrix implant held in the forceps. The actual size of the sponge is 5 mm in diameter and 1.5 mm of thickness. FIG. 2B is a longitudinal scheme of a honeycomb structure of an acellular matrix implant showing a relative localization of collagen sponge and porous collagen gel wherein the pore size is between 200 and 400 μm.

FIG. 3 shows a micrograph of the two control empty defect sites A and B (4 mm in diameter and 1-1.5 mm in depth) created on the weight-bearing site of the swine medial femoral condyle.

FIG. 4 is a micrograph of the two defect sites A and B generated on the weight-bearing site of the swine medial femoral condyle, implanted with acellular matrix implants. The defect has 4 mm in diameter and 1-1.5 mm in depth. The implants have 5 mm diameter and 1.5 mm thickness. Each implant is sutured using 4 absorbable sutures and two non-absorbable sutures. The bottom of the defect is lined up with a first protective biodegradable polymer barrier and the implant is covered with the second protective biodegradable polymer barrier.

FIG. 5 shows arthroscopic evaluation of a magnified empty defect 2 weeks after defect creation showing the defect to be fully exposed and empty.

FIG. 6 shows arthroscopic evaluation of a magnified defect treated with the acellular matrix implant 2 weeks after the defect creation. The superficial cartilage layer overgrowing the implant site forms a smooth flat surface over the defect.

FIG. 7 is a graph illustrating a histological grading of the repair tissue.

FIG. 8A shows a histological evaluation (29× magnification) of the empty defect (D) at a control site (A). FIG. 8B shows a higher (72×) magnification of the defect site (D). The defect is surrounded by the host cartilage (H) with underlying subchondral bone (SB) area. Fibrous tissue (F) formation is seen in both figures at the empty defect site. Fibrovascular pannus (F) is formed at empty defect site as indicated by the absence of the S-GAG accumulation.

FIG. 9A shows a histological evaluation (29× magnification) of the empty defect (D) at a control site (B). FIG. 9B shows a higher (72×) magnification of the defect site (D). The defect is surrounded by the host cartilage (H) with underlying subchondral bone (SB) area. Fibrous tissue (F) formation is seen in both FIGS. 9A and 9B at the empty defect site with slight accumulation of S-GAG accumulation.

FIG. 10A shows a histological evaluation (29× magnification) of the acellular implantation (I) at the implant site (A). FIG. 10B shows acellular implantation at higher (72×) magnification of the implant site (I). The implant site is surrounded by the host cartilage (H) with underlying subchondral bone (SB) area. Superficial cartilage layer is shown to cover the implant site. In both FIGS. 10A and 10B normal S-GAG accumulation and formation of hyaline-like cartilage was observed at the implant site.

FIG. 11A shows a histological evaluation (29× magnification) of the acellular implantation (I) at the implant site (B). FIG. 11B shows acellular implantation at higher (72×) magnification of the implant site (I). The implant site is surrounded by the host cartilage (H) with underlying subchondral bone (SB) area. Superficial cartilage layer is shown to cover the implant site. In both FIGS. 11A and 11B normal S-GAG (*) accumulation and formation of hyaline-like cartilage was observed at the implant site.

FIG. 12 illustrates a degradation pattern in vivo, at three months after the acellular matrix implantation, of the second protective biodegradable polymer placed over the implant. The newly formed superficial cartilage layer is overgrowing the implant. FIG. 12 clearly shows the second protective biodegradable polymer barrier as partially degraded at three months after the implantation. FIG. 12A shows a surface view of the Safranin-O stained implantation site. FIG. 12B shows a side view of the Safranin-O stained implantation site. FIG. 12C shows the bottom view of the Safranin-O stained implantation site. FIG. 12D shows a surface view of the protective biodegradable polymer barrier immunostaining. FIG. 12E shows a side view of the protective biodegradable polymer barrier immunostaining. FIG. 12F shows a bottom view of the protective biodegradable polymer barrier immunostaining. Safranin-O staining, seen as reddish color, indicates S-GAG accumulation. Brown color indicates remaining polymer in samples processed immunohistochemicallly.

FIG. 13 shows an example image of a full thickness defect (D) after harvest created at femoral condyle of mini-pig at 72× magnification. Surrounding host cartilage (H), subchondral bone area (SB) and remaining calcified cartilage area are also indicated.

FIG. 14 illustrates toxicity of the unmodified sealant CT3 and CT3 sealant modified to a substantially non-toxic biodegradable polymer having an extended polymerization time deposited into porcine femoral condyle, compared to untreated intact controls. FIG. 14A (side view) and FIG. 14B (bottom view) show distribution of the living cells, stained green, and dead cells, stained red, from both views. FIG. 14C (side view) and FIG. 14D (bottom view) show side and bottom view distribution of the living (green) and dead (red) cells observed after use of unmodified CT3 at pH 3.4 used as a bottom polymer barrier. FIG. 14E (side view) and FIG. 14F (bottom view) show side and bottom view distribution of the living (green) and dead (red) cells observed after use of CT3 modified with a buffer to pH 6.5, used as a bottom polymer barrier. FIG. 14G (side view) and FIG. 14H (bottom view) show side and bottom view distribution of the living (green) and dead (red) cells observed after use of CT3 modified with a buffer to pH 7.0, used as a bottom polymer barrier. FIG. 14I (side view) and FIG. 14J (bottom view) show side and bottom view distribution of the living (green) and dead (red) cells observed after use of the CT3 modified with a buffer to pH 7.5, used as a bottom polymer barrier.

FIG. 15 show results of the lap shear test in biodegradable polymers modified as shown in FIGS. 14A-14J.

### DEFINITIONS

As used herein:

“Acellular” means an implant lacking any biologically active cells.

“Acellular matrix implant” or “acellular implant” means a biologically acceptable implant whether in the form of collagenous sponge, collagenous honeycomb, collagenous scaffold, thermo-reversible gelation hydrogel, a polymer of an aromatic organic acid or an absorbable caprolactone, polymer without any biologically active cells, forming a matrix into which the chondrocytes may migrate.

“Articular cartilage” means a hyaline cartilage of the joints, such as the knee joint.

“Subchondral” means a bone underlying the joint cartilage.

“Subchondral bone” means a very dense, but thin layer of bone just below the zone of calcified cartilage and above the cancellous or trabecular bone which forms the bulk of the bone structure of the limb.

“Osteochondral” means combined area of the cartilage and bone where a lesion or lesions occur.

“Osteochondral defect” means a lesion which is a composite lesion of cartilage and underlying bone.

“Bone defect” or “bone lesion” means the defect which is localized under the subchondral bone region and is thus a defect/lesion in a skeletal bone.

“Osteoblast” means a bone forming cell.

“Chondrocyte” means a nondividing cartilage cell which occupies a lacuna within the cartilage matrix.

“Support matrix” means biologically acceptable sol-gel or collagenous sponge, scaffold, honeycomb, hydrogel, a polymer of an aromatic organic acid or caprolactone suitable for receiving activated migrating chondrocytes or osteocytes that provides a structural support for growth and three-dimensional propagation of chondrocytes and for formulating of new hyaline cartilage or for migration of osteochondrocytes into the bone lesions. The support matrix is prepared from such materials as Type I collagen, Type II collagen, Type IV collagen, gelatin, agarose, cell-contracted collagen containing proteoglycans, glycosaminoglycans or glycoproteins, polymers of aromatic organic acids, fibronectin, laminin, bioactive peptide growth factors, cytokines, elastin, fibrin, synthetic polymeric fibers made of poly-acids such as polylactic, polyglycolic or polyamino acids, polycaprolactone, absorbable epsilon caprolactone polymer, polypeptide gel, copolymers thereof and combinations thereof. The gel solution matrix may be a polymeric thermo-reversible gelling hydrogel. The support matrix is preferably biocompatible, biodegradable, hydrophilic, non-reactive, has a neutral charge and is able to have or has a defined structure.

“Mature hyaline cartilage” means cartilage consisting of groups of isogenous chondrocytes located within lacunae cavities which are scattered throughout an extracellular collagen matrix.

“Protective biodegradable polymer barrier”, “biodegradable polymer barrier”, “biodegradable polymer” or “protective polymer” means a biologically acceptable biocompatible, substantially non-toxic polymerizing formulation having an extended polymerization time between at least two minutes and no more than ten minutes, preferably between about three minutes and five minutes. The protective biodegradable polymer barrier is thus a biologically acceptable synthetic or natural polymer composition which is biodegradable in time, has adhesive or cohesive properties, and is typically a derivatized polyethylene glycol (PEG) preferably cross-linked with a collagen compound, typically alkylated collagen. Examples of suitable derivatized polyethylene glycol are tetra-hydrosuccinimidyl or tetra-thiol derivatized PEG, or a combination thereof, commercially available from Cohesion Technologies, Palo Alto, Calif. under the trade name CoSeal™, described in J. Biomed. Mater. Res. (Appl. Biomater.), 58:545-555 (2001), herein incorporated by reference, or two-part polymer compositions comprising polyalkylene oxide and/or polyethylene glycol wherein the two parts are cross-linked through a covalent bond, as described in U.S. Pat. Nos. 6,312,725B1 and 6,624,245B2, both herein incorporated by reference, and further cross-linked with methylated collagen, such as a cross-linked succinimidyl and thiol-derivatized polyethylene glycol with methylated collagen, wherein the resulting composition is modified to have an extended polymerization time between at least 2 to 10 minutes, required by the method of the current invention. The unmodified compositions described in the cited references typically gel rapidly, upon contact with tissue, particularly with tissue containing collagen, such gelation occurring within 5 to 60 seconds.

“Substantially” means that only a few, if any, dead cells appear after the use of the modified biodegradable polymer.

“Collagen” means and includes all forms of collagen including those which have been modified or chemically or biologically processed and purified, intact or denatured. The collagen most preferred for the use in this invention is a substantially pure collagen having removed substantially all impurities and immunogenic substances. Particularly preferred is a recombinantly made collagen.

“Recombinant collagen” means a collagen produced recombinantly by the methods described, for example in the U.S. Pat. No. 5,667,839, herein incorporated by reference.

“Bottom protective biodegradable polymer barrier” or “first protective biodegradable polymer barrier” means a biologically acceptable tissue protective biodegradable polymer barrier which is nontoxic to cells, modified as defined above, which is deposited at the bottom of the lesion. In case of the osteochondral defect, the first protective biodegradable polymer barrier is deposited over the bone-inducing composition or a carrier comprising said composition deposited into the bone lesion effectively sealing, separating and protecting the bone lesion from chondrocyte migration as well as protecting the cartilage lesion from migration of osteocytes.

“Top protective biodegradable polymer barrier” or “second protective biodegradable polymer barrier” means a biologically acceptable protective biodegradable polymer barrier which is substantially non-toxic to cells or tissue, modified as defined above, which is deposited above and over the acellular matrix implant implanted into a lesion and may promote formation of the superficial cartilage layer. The second (top) protective biodegradable polymer barrier may or may not be the same as the first (bottom) protective biodegradable polymer barrier.

“Modified protective biodegradable polymer barrier”, “modified biodegradable polymer barrier”, “modified biodegradable polymer” or “modified protective polymer” means any suitable protective biodegradable polymer barrier which does not show cell or tissue toxicity for use in the invention modified to have a polymerization time of at least two minutes and no longer than ten minutes, typically achieved by change in ratio of the buffer and/or the acid adjustment of the composition pH to values about or lower than pH 7.5.

“Bone-inducing composition” or “a carrier comprising said composition” means a composition comprising at least one bone-inducing agent or, preferably, a combination of several agents, typically dissolved in a carrier or incorporated into a matrix similar to the acellular matrix implant.

“Bone-inducing carrier”, “carrier comprising bone-inducing composition” or “bone acellular implant” means any carrier which contains bone-inducing agents and which by itself promotes bone formation or is suitable for depositing said bone-inducing composition comprising at least one bone-inducing agent or, preferably, a combination of several agents. Typically, the carrier will be an acellular biodegradable porous matrix, hydrogel, sponge, honeycomb, scaffold or a polymer of an aromatic organic acid structure having large pores from about 50 to about 150 μm, which pores encourage migration of osteoblast and interconnecting small pores of about 0.1 to about 10 μm which promote support and encourage formation of bone. The surface of such carrier might be negatively charged encouraging pseudopod attachment of osteoblasts and subsequent bone formation. One example of the suitable carrier promoting bone formation is a polymer of an aromatic organic acid with controllable degree of degradation which is sufficiently hard but has a sponge-like structure. “Bone-inducing agents” means agents which induce, support or promote bone growth and repair of bone defects. Exemplary bone-inducing agents are calcium phosphate, hydroxyapatite, organoapatite, titanium oxide, demineralized bone powder, poly-L-lactic and polyglycolic acid or a copolymer thereof or a bone morphogenic protein, among others.

“De novo” or “de novo formation” means the new production of cells, such as chondrocytes, fibroblasts, fibrochondrocytes, tenocytes, osteoblasts and stem cells capable of differentiation, or tissues such as cartilage connective tissue, hyaline cartilage, fibrocartilage, tendon, and bone within a support structure, such as multi-layered system, scaffold or collagen matrix or formation of superficial cartilage layer.

“Superficial cartilage layer” means an outermost layer of cartilage that forms the layer of squamous-like flattened superficial zone chondrocytes covering the layer of the second protective biodegradable polymer barrier and overgrowing the lesion.

“Thermo-reversible” means a compound or composition changing its physical properties such as viscosity and consistency, from sol to gel, depending on the temperature. The thermo-reversible composition is typically completely in a sol (liquid) state at between about 5 and 15° C. and in a gel (solid) state at about 25-30° C. and above. The gel/sol state in between shows a lesser or higher degree of viscosity and depends on the temperature. When the temperature is higher than 15° C., the sol begins to change into gel and with the temperature closer to 30-37° the sol becomes more and more solidified as gel. At lower temperatures, typically lower than 15° C., the sol has more liquid consistency.

“TRGH” means thermo-reversible gelation hydrogel material in which the sol-gel transition occurs on the opposite temperature cycle of agar and gelatin gels. Consequently, the viscous fluidic phase is in a sol stage and the solid phase is in a gel stage. TRGH has very quick sol-gel transformation which requires no cure time and occurs simply as a function of temperature without hysteresis. The sol-gel transition temperature can be set at any temperature in the range from 5° C. to 70° C. by molecular design of thermo-reversible gelation polymer (TGP), a high molecular weight polymer of which less than 5 wt % is enough for hydrogel formation.

“Sol-gel solution” means a colloidal suspension which, under certain conditions, transitions from a liquid (sol) to a solid material (gel). The “sol” is a suspension of aqueous collagen that is transitioned, by heat treatment, into a gel.

“GAG” means glycosaminoglycan.

“S-GAG” means sulfated glycosaminoglycan.

“Aggrecanase” means aggrecanase enzyme.

“Cathepsin” means a proteinase or peptidase enzyme.

“MMP” means matrix metalloproteinase, an enzyme associated with cartilage degeneration in an injured or diseased joint.

“DMB” means dimethylene blue used for staining of chondrocytes.

“Superficial zone cartilage” means the flattened outermost layer of chondrocytes covering the extracellular matrix intermediate zone and deeper zone of mature articular cartilage in which non-dividing cells are dispersed.

“Connective tissue” means tissue that protect and support the body organs, and also tissues that hold organs together. Examples of such tissues include mesenchyme, mucous, connective, reticular, elastic, collagenous, bone, blood, or cartilage tissue such as hyaline cartilage, fibrocartilage, and elastic cartilage.

“Adhesive strength” means a peel bond strength measurement, which can be accomplished by bonding two plastic tabs with an adhesive formulation. The tabs can be formed by cutting 1×5 cm strips from polystyrene weighing boats. To the surface of the boat are bonded (using commercial cyanoacrylate Superglue), sheets of sausage casing (collagen sheeting, available from butcher supply houses). The sausage casing is hydrated in water or physiological saline for 20 min to one hour and the adhesive is applied to a 1×1 cm area at one end of the tab; the adhesive is cured. Then, the free ends of the tab are each bent and attached to the upper and lower grips, respectively, of a tensile testing apparatus and pulled at 10 mm/min strain rate, recording the force in Newtons to peel. A constant force trace allows estimation of N/m, or force per width of the strip. A minimum force per width of 10 N/m is desired; 100N /m or higher is more desirable. Alternatively, the same tab can be bonded (a single tab) over a 1×1 cm area to tissue, either dissected or exposed tissue in a living animal, during surgery. The free end of the tab is then gripped or attached through a perforation to a hook affixed to a hand-held tensile test device (Omega DFG51-2 digital force gauge; Omega Engineering, Stamford, Conn.) and pulled upward at approximately 1 cm/sec. The maximum force required to detach the tab from the tissue is recorded. The minimum force desired in such measurements would be 0.1 N to detach the tab. Forces or 0.2 to 1 N are more desirable. “Cohesive strength” means the force required to achieve tensile failure and is measured using a tensile test apparatus. The glue or adhesive can be cured in a “dog-bone”-shaped mold. The wide ends of the formed solid adhesive can then be affixed, using cyanoacrylate (Superglue) to plastic tabs, and gripped in the test apparatus. Force at extensional failure should be at least 0.2 MPa (2 N/cm²) but preferably 0.8 to 1 MPa or higher.

“Lap shear measurements” means a test of bonding strength, in which the protective biodegradable polymer barrier formulation is applied to overlapping tabs of tissue, cured, and then the force to pull the tabs apart is measured. The test reflects adhesive and cohesive bonding; strong adhesives will exhibit values of 0.5 up to 4-6 N/cm² of overlap area.

### DETAILED DESCRIPTION OF THE INVENTION

This invention is based on findings that when a biodegradable acellular matrix implant is deposited into a lesion of injured, traumatized, aged or diseased cartilage between two layers of a biodegradable polymer barrier, the acellular matrix implant promotes generation of a new extracellular matrix ultimately resulting in formation of a healthy hyaline cartilage rather than fibrocartilage. Additionally, the invention is based on findings that in case of the osteochondral defect, when the acellular implant is deposited in a cartilage lesion in conjunction with depositing a bone-inducing composition into a bone defect, both the bone and cartilage can be repaired independently.

The invention thus, in its broadest scope, concerns a method for repair and restoration of damaged, injured, traumatized or aged cartilage or for repair of osteochondral defects and restoration of both the cartilage and bone into their full functionality by implanting, during arthroscopic surgery, an acellular matrix implant and/or depositing a bone-inducing composition or a carrier comprising said composition into the bone lesion before implanting the acellular matrix implant into the cartilage lesion. The invention further includes a method for fabrication of said acellular matrix implant, preparation of said bone-inducing composition or a carrier comprising said composition and a method for preparation of protective biodegradable polymers having a polymerization time between about 2 and 10 minutes.

Briefly, for treatment of the articular lesions, the invention comprises preparation of the acellular matrix implant for implanting into a joint cartilage lesion, said implant comprising a collagenous, thermo-reversible gel, an aromatic organic acid or absorbable epsilon-caprolactone polymer support matrix in two or three-dimensions. The acellular matrix implant may contain various supplements, such as matrix remodeling enzymes, metalloproteinases (MMP-9, MMP-2, MMP-3), aggrecanases, cathepsins, growth factors, donor's serum, ascorbic acid, insulin-transferrin-selenium (ITS), etc.

For treatment of osteochondral defects, the invention comprises preparation of a bone-inducing composition, or a carrier comprising said composition, said composition comprised of bone-inducing agents, such as demineralized bone powder, calcium phosphate, hydroxyapatite, organoapatite, titanium oxide, poly-L-lactic and polyglycolic acid or a copolymer thereof, alone or in combination, or a bone morphogenic protein, depositing said composition into the bone lesion and covering said bone-inducing composition or a carrier comprising said composition with a protective biodegradable polymer barrier followed by depositing said acellular matrix implant into the cartilage lesion and covering said implant with the second protective biodegradable polymer barrier.

The acellular matrix implant is implanted into a cartilage lesion cavity formed by two, first and second, layers of adhesive protective biodegradable polymers. However, in certain circumstances, the acellular matrix implant may be also deposited into the cartilage lesion without either the bottom or top protective biodegradable polymer barrier or without both protective biodegradable polymer barriers.

When the protective biodegradable polymer barriers are used, which is preferred, in the method for repair of cartilage, the first (bottom) layer of the protective biodegradable polymer is deposited at and covers the bottom of the cartilage lesion. Its function is to protect the integrity of said lesion from cell migration and from effects of various blood and tissue debris and metabolites and also to form a bottom of the cavity into which the acellular matrix implant is deposited. The first layer of the protective biodegradable polymer may also become a covering layer deposited over the bone-inducing composition or a carrier comprising said composition placed into the bone lesion within the subchondral bone area.

Studies of induced defects of the pig's femoral condyle confirmed that implantation of a biodegradable acellular matrix implant into a cartilage lesion of which a bottom was covered with a layer of a protective biodegradable polymer non-toxic to cells having an extended polymerization time, combined with an implantation procedure disclosed herein and performed under defined conditions, results in formation of the new extracellular matrix (ECM) within the lesion at the injured site leading to generation of the hyaline cartilage. Similarly, a deposition of a bone-inducing composition comprising bone-inducing agents, or a carrier comprising said composition, into the bone defect promotes natural healing of bone by inducing migration of osteoblasts into said bone lesion and, combined with the acellular matrix implant as described above, leads to healing and reconstruction of both the bone and cartilage.

The method for using the acellular matrix implant for generation of the hyaline cartilage is particularly suitable for treatment of lesions in younger patients with focused lesions where the cartilage has not developed an incipient osteoarthritic conditions, that is in patients who would typically be treated with microfracture or with cleaning the articular cartilage in the joint, such as in, for example, arthroscopic surgery following a sports injury. Such patients stand a high probability of restoring a fully functional hyaline cartilage, or in case of osteochondral defects, a fully functional cartilage and bone, without need of and aggravation associated with undergoing additional one or multiple surgeries.

One advantage of using the above-described method is that the acellular matrix implant and/or the bone-inducing composition or a carrier comprising such composition is non-immunogenic as it does not involve any biological material, can be pre-manufactured well before the operation and can be introduced during the first arthroscopy, when the diagnosis, cleaning and debridement of the lesion takes place without a need for further biopsy, cell culturing, additional surgeries or treatments to prevent immune reactions. Undivisible part of the method are two protective barriers emplaced at the bottom and at the top of the cartilage or osteochondral lesions. The surgical restrictions require that these two barriers are biologically acceptable, substantially non-toxic to cells or tissue, fully biodegradable and removed naturally from the site of the lesion within time of approximately 2-3 months following the surgery and that they are polymerizable at physiologically acceptable pH, within a specifically defined time between not less than 2 minutes and at most 10 minutes with a preferred polymerization time between 3 and 5 minutes.

I. Cartilage, Bone and Properties Thereof

Cartilage and bone, both, are connective tissues providing support in the body for other soft tissues.

Bone is a hard connective tissue forming a skeleton, consisting of osteoblast cells embedded in a matrix of mineralized ground substance and collagen fibers. The collagen fibers are impregnated with a form of calcium phosphate similar to hydroxyapatite as well as with substantial quantities of carbonate, citrate, sodium and magnesium. Bone is composed of approximately 75% of inorganic material and 25% of organic material. Bone consists of a dense outer layer of compact substance covered by periosteum and an inner, loose spongy substance, i.e. bone marrow. Bone emplaced immediately below the cartilage is called subchondral bone.

Cartilage is a mature connective tissue covering joints and bones which is comprised of metabolically active but non-dividing chondrocytes. This results in essential non-existence of spontaneous ability of the cartilage to self-repair following the injury or damage caused by age or disease.

Cartilage is characterized by its poor vascularity and a firm consistency, and consists of mature non-dividing chondrocytes (cells), collagen (interstitial matrix of fibers) and a ground proteoglycan substance (glycoaminoglycans or mucopolysaccharides). The later two are cumulatively known as extracellular matrix.

There are three kinds of cartilage, namely hyaline cartilage, elastic cartilage and fibrocartilage. Hyaline cartilage, found primarily in joints, has a frosted glass appearance with interstitial substance containing fine type II collagen fibers obscured by proteoglycan. Elastic cartilage is a cartilage in which, in addition to the collagen fibers and proteoglycan, the cells are surrounded by a capsular matrix further surrounded by an interstitial matrix containing elastic fiber network. The elastic cartilage is found, for example, in the central portion of the epiglottis. Fibrocartilage contains Type I collagen fibers and is typically found in transitional tissues between tendons, ligaments or bones and also as a low quality replacement of injured hyaline cartilage. This invention utilizes properties of acellular matrix implant combined with certain conditions existing naturally in the surrounding native cartilage further combined with certain steps according to the method of the invention, to achieve the full healing and replacement of injured cartilage with the healthy and functional hyaline cartilage.

A. Articular Cartilage and Articular Cartilage Defects

The articular cartilage of the joints, such as the knee cartilage, is hyaline cartilage which consists of approximately 5% of chondrocytes (total volume) seeded in approximately 95% extracellular matrix (total volume) . The extracellular matrix contains a variety of macromolecules, including collagen and glycosaminoglycan (GAG). The structure of the hyaline cartilage matrix allows it to reasonably well absorb shock and withstand shearing and compression forces. Normal hyaline cartilage has also an extremely low coefficient of friction at the articular surface.

Healthy hyaline cartilage has a contiguous consistency without any lesions, tears, cracks, ruptures, holes or shredded surface. Due to trauma, injury, disease such as osteoarthritis, or aging, however, the contiguous surface of the cartilage is disturbed and the cartilage surface shows cracks, tears, ruptures, holes or shredded surface resulting in cartilage lesions.

The articular cartilage is an unique tissue with no vascular, nerve, or lymphatic supply. The lack of vascular and lymphatic circulation may be one of the reasons why articular cartilage has such a poor, almost non-existent intrinsic capacity to heal. The mature metabolically active but non-dividing chondrocytes in their lacunae surrounded by extracellular matrix do not respond to damage signals by generating high-quality hyaline cartilage. After a significant injury, unique mechanical functions of articular cartilage are never reestablished spontaneously and never completely because the water-absorption capacity of the type II collagen/proteoglycan network is disturbed. The usual replacement material for hyaline cartilage, which might develop spontaneously in response to the injury of hyaline cartilage and which replaces the injured cartilage, is the much weaker and functionally inferior fibrocartilage.

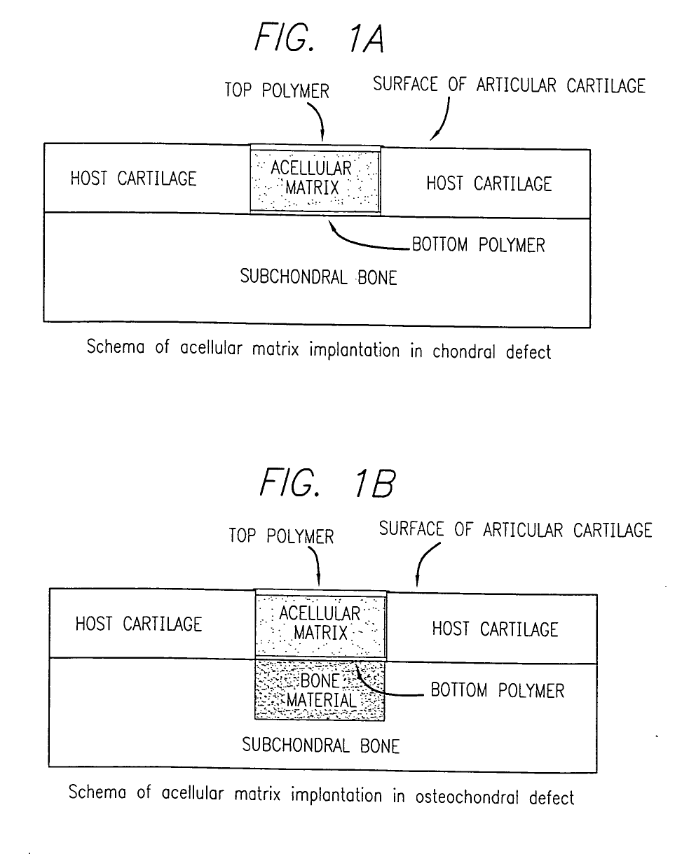

Defects occurring due to cartilage trauma, injury, disease or aging are tears, cracks, ruptures or holes which are solely located in the joint cartilage. According to the method of the invention, when such defect is treated, the implant is deposited within the lesion, as illustrated in FIG. 1A.

FIG. 1A is a schematic representation of an acellular matrix implant implanted into the cartilage defect. The scheme shows the lesion implantation site with acellular matrix implanted therein surrounded by host cartilage with underlaying undisturbed subchondral bone. Emplacement of the top and bottom protective biodegradable polymer barriers are also illustrated.

B. Currently Available Procedures for Repair of Cartilage

A variety of surgical procedures have been developed and used in attempts to repair damaged cartilage. These procedures are performed with the intent of allowing bone marrow cells to infiltrate the defect and promote its healing. Generally, these procedures are only partly, if at all, successful. More often than not, these procedures result in formation of a fibrous cartilage tissue (fibrocartilage) which does fill and repair the cartilage lesion but, because it is qualitatively different being made of Type I collagen fibers, it is less durable, less resilient and generally inferior than the normal articular hyaline cartilage and thus has only a limited ability to withstand shock and shearing forces than does healthy hyaline cartilage. Since all diarthroid joints, particularly knee joints, are constantly subjected to relatively large loads and shearing forces, replacement of the healthy hyaline cartilage with fibrocartilage does not result in complete tissue repair and functional recovery.

Among the currently available procedures for repair of the articular cartilage injuries are the microfracture technique, the mosaicplasty technique and autologous chondrocyte implantation (ACI). However, in one way or another, all these techniques are problematic. The mosaicplasty technique and ACI, for example, need a biopsy of cartilage from a non-damaged articular cartilage area and subsequent cell culture to grow the number of cells. As a consequence, these techniques require at least two separate surgeries and because of the presence of a biological material, they carry with it a potential risk of immunological reactions. One system, the Carticel® system additionally requires a second surgery site to harvest portion of and, therefore, disrupt, tibial periosteum. While the microfracture technique does not require a biopsy of articular cartilage, the resulting tissue which develops is always fibrocartilage.

The method for treatment of injured, traumatized, diseased or aged cartilage according to the current invention obviates the above problems as it comprises treating the injured, traumatized, diseased or aged cartilage with an acellular matrix implant without need to remove tissue or cells for culturing and thus consequently without any biological material present, said implant prepared by methods described below and implanted into the cartilage lesion during the debriding surgery, as described below.

C. Osteochondral Area and Osteochondral Defects

Osteochondral area, in this context, means an area where the bone and cartilage connect to each other and where the osteochondral defect, that is a lesion through both tissues, often occurs during the injury.

FIG. 1B is a schematic representation of implantation of an acellular matrix implant in the osteochondral defect. The scheme shows the cartilage lesion implantation site with the acellular matrix implanted therein surrounded by host cartilage with underlaying bone lesion in the subchondral bone. A bone-inducing composition or an acellular implant carrier comprising said composition is deposited into the bone lesion separated from the cartilage lesion by the bottom protective biodegradable polymer barrier. Emplacement of the top and bottom protective biodegradable polymer barriers illustrates separation of the bone lesion from the cartilage lesion by the bottom protective biodegradable polymer barrier such that each the cartilage lesion and the bone lesion are treated separately using different means, namely the acellular matrix implant for treatment of the cartilage lesion and the bone-inducing composition or the acellular carrier comprising said composition for treatment of the bone defect.

Osteochondral defects are thus defects that are composites of cartilage and underlying bone. Up-to-date, commonly used treatments for osteochondral defects are surgical excisions, mosaicplasty, osteochondral autogenous grafting, allogenic grafting, bone cementing, deposition of metal or ceramic solid composite materials, porous biomaterials and, lately, a transplantation of autologous chondrocytes. However, since the main objective of all these treatments is restoration and maintaining of the supporting and mechanical function of the bone, regretfully, none of these procedures was found to be successful in treating these defects and safe or comfortable for a patient. Typically, these procedures involve two or more surgical procedures and long period, generally at least two to three weeks, of time to culture the transplantable cells. For example, mosaicplasty requires removal of circular pieces of healthy subchondral bone and cartilage to be used as transplantable plugs at a defect site. One obvious problem with mosaicplasty is that the surgeon, in an open surgery, is disrupting healthy tissue in order to repair the subchondral defect. Clearly, the multiple surgeries and long period of time between them necessarily extend a time of recovery to fully functional joint and often result only in a partial functional restoration as both the bone and cartilage defects are filled with the fibrocartilage instead of the bone and hyaline cartilage.

One example of the osteochondral defect which is common and very difficult to treat is osteochodritis dissecans. Osteochodritis dissecans is a focal bone-cartilage lesion characterized by separation of an osteochondral fragment from the articular surface. Attempts to treat this injury with allograph transplants faces the same problem of the second surgery and disruption of the healthy tissue, as described above. Thus it would be advantageous to have available a method which would remove a need for second surgery and yet provide a means for a cartilage and bone repair.

The current method provides a solution to the above-outlined problems by implanting, during the first arthroscopic surgery, a bone-inducing composition or a carrier comprising said composition comprising a bone-inducing agents into the bone lesion insulated from the cartilage lesion by a layer of a biodegradable polymer barrier and then implanting an acellular matrix implant into the cartilage lesion covered with a second layer of the biodegradable polymer barrier effectively insulating the implant from the outside environment, thereby providing, in one surgery, treatments for both the bone and cartilage defects.

II. An Acellular Matrix Implant for Treatment of Cartilage Lesions

The current invention provides a method for treatment of injured, damaged, diseased or aged cartilage. To this end, the method involves implantation of the acellular matrix implant into the injured, damaged, diseased or aged cartilage lesion at a site of injury or at a site of a defect caused by disease or age, in a single surgery. The acellular matrix implant is a collagenous or non-collagenous construct, such as a construct prepared from polymer of an aromatic organic acid or an absorbable caprolactone polymer, comprising various components as described below.

A. Preparation of an Acellular Matrix Implant

Preparation of the acellular matrix implant for implanting into the cartilage lesion involves preparation of acellular support matrix, typically a collagenous scaffold or sponge, thermo-reversible gelation hydrogel, a construct made of polymer of an aromatic organic acid or of an absorbable caprolactone polymer and implanting said matrix into the cartilage defect in situ.

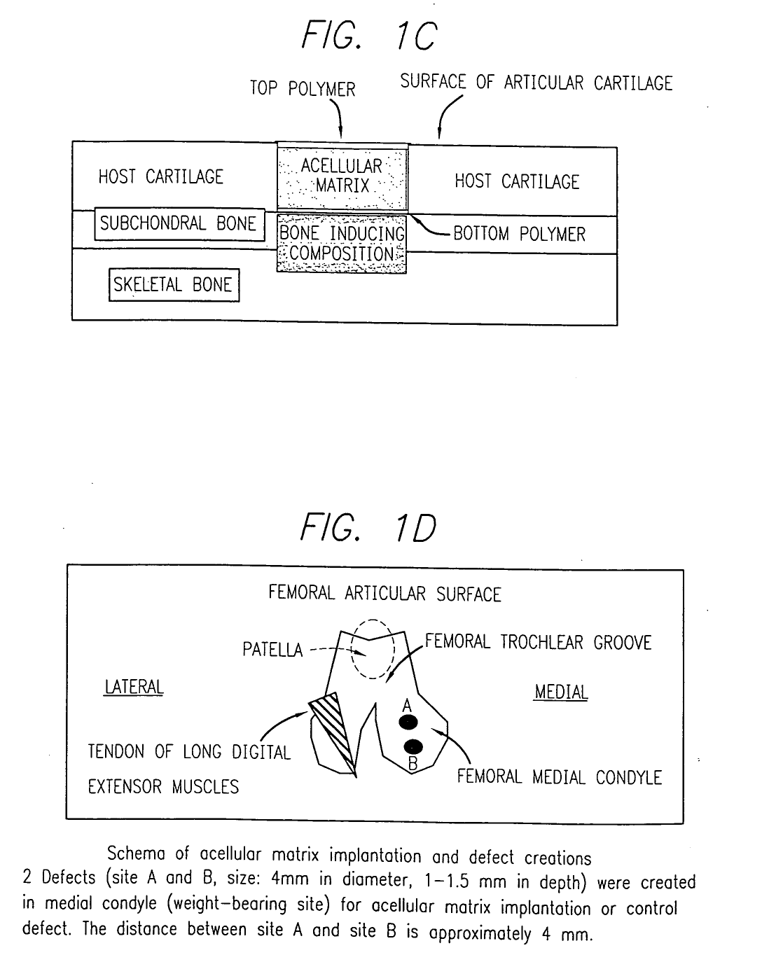

The acellular matrix implant, such as the one seen in FIG. 2A, is prepared according to the method of the invention and implanted into artificially generated lesions in a swine's knee weight bearing region. FIG. 2A is an image of an actual acellular matrix sponge implant used for implantation, here held in the forceps. The sponge has a size of 5 mm in diameter and 1.5 mm in thickness and comprises a composition of collagen sponge and collagen gel having pores of sizes from about 200-400 μm (FIG. 2B). When the sponge is implanted into the lesion, chondrocytes are activated and migrate into the porous structure of the sponge where they begin to secrete a new extracellular matrix ultimately replacing the collagen sponge and gel with the new hyaline cartilage. The sponge and gel naturally biodegrade and are metabolically removed from the lesion.

FIG. 2B is a cross-side view scheme of a honeycomb structure of the acellular matrix sponge seen in FIG. 2A illustrating a relative positioning of the collagen sponge, collagen gel and pores within the acellular matrix sponge.

The matrices of the acellular matrix implant deposited into the lesion are comprised of biodegradable materials which permit said implant to function for certain period of time needed for formation of the hyaline cartilage. Such biodegradable materials are subsequently biodegraded and metabolically removed from the site of implantation leaving, if any, only non-toxic residues. These matrices are covered with a second layer of a biodegradable polymer barrier covering and insulating the matrix from external environment until the superficial cartilage layer is formed. The superficial cartilage layer then covers the lesion containing the implant thereby protecting a newly formed hyaline cartilage and essentially takes over the protective and insulating function of the biodegradable polymer which at that time is either completely or partially biodegraded.

The above-described matrices may additionally incorporate enzymes, such as metalloproteinases, paracrine or autocrine growth hormones, GAG-lyases and such like enzymes, soluble protein mediators and other modulators and supplements. Presence or addition of these materials may enhance activation of mature, metabolically active but non-dividing chondrocytes present in the surrounding native host cartilage and migration of these chondrocytes from the native host cartilage surrounding the lesion cavity into said acellular matrix implant emplaced within said lesion.

The present invention thus concerns a discovery that when the acellular matrix implant according to the invention is implanted into a cartilage defect over the non-toxic biodegradable polymer barrier deposited at the bottom of the lesion and under the non-toxic biodegradable polymer barrier deposited over the implant, under conditions described below, the older inactive chondrocytes residing within the surrounding native cartilage are induced to migrate into the defect where these chondrocytes are activated from static non-dividing stage to an active stage where they divide, multiply, promote growth of the extracellular matrix and generate a new hyaline cartilage in situ. Following the implantation of the acellular matrix implant, the cartilage defect is quickly repaired, particularly in the young individuals, by chondrocyte migration and by formation of the extracellular matrix supported by the metalloproteinases naturally present in sufficient amounts in tissues of the young individuals. For the repair of lesions in older subjects, the GAG-lyases and metalloproteinases, growth factors and other components are added or incorporated into said matrix before implantation or they may be conveniently used to coat said matrix to promote degradation of the injured cell.

A process for activation of chondrocytes was found to require certain period of time, typically from about 1 hour to about 3 weeks, typically only about 6 hours to about 3 days. The process for complete replacement of the implant matrix with the hyaline cartilage typically takes from one week to several months provided that the treated individual becomes normally physically active subjecting said new cartilage to the intermittent hydrostatic pressure by, for example, walking, running or biking.

The acellular implant, including the bottom and top polymer barriers are fully biodegradable and are removed from the lesion site by natural metabolic processes without leaving any detrimental residua within about 2-4 months following their implantation.

B. Induction of Chondrocyte Migration

Induction of chondrocyte migration from the surrounding native cartilage involves biological actions of various agents either naturally present within the cartilage, cartilage surrounding tissue, blood or plasma or are added either before, during or after the surgery to promote release, activation and migration of chondrocytes from the native surrounding host cartilage into the implant.

One of the steps in achieving the activation of the chondrocytes is the use of the two protective substantially non-toxic biodegradable polymer barriers, one at the top and one at the bottom, of the articular cartilage lesion. This step results in creation of a cavity into which the acellular matrix implant is deposited and in insulation and protection of the implant integrity from cellular debris, blood cells, metabolites and other undesirable contaminants. However, since the protective barriers are placed at the bottom and over the top of the implant, the sides of the lesion remain open toward the host's surrounding healthy cartilage and permit migration of chondrocytes, infusion and concentration of soluble protein mediators, modulators, enzymes, growth or other factors, etc., naturally present in the host's surrounding healthy cartilage, into the acellular matrix implant.

Insulating the top and bottom of the defect by the two non-toxic biodegradable polymer barriers before and after insertion of the acellular matrix implant results in accumulation of autocrine and paracrine growth factors that are released by chondrocytes in the adjacent extracellular matrix, enabling these factors to induce cell migration into the implant. Suitable growth factors include, among others, certain transforming growth factors, platelet-derived growth factors, fibroblast growth factors and insulin-like growth factor-I. Additionally, these and other supplements, such as the GAG-lyases (matrix remodeling enzymes), may be used to coat the implant before its insertion into the lesion.

The acellular matrix implant sequestered within the lesion cavity by the top and bottom protective biodegradable polymer barrier, as described above, remains in flowable communication with the adjacent cartilage. This arrangement creates conditions resulting in decrease of levels of inhibitors of the matrix remodeling enzymes, such as tissue inhibitors of metalloproteinase-1 (TIMP-1), metalloproteinase-2 (TIMP-2) and metalloproteinase-3 (TIMP-3), at the defect site. As a consequence, the matrix metalloproteinases (MMP-1, MMP-2, MMP-3) become accessible to enzymatic activation and degrade the adjacent extracellular matrix thereby releasing chondrocytes localized therein resulting in chondrocytes migration from the surrounding host cartilage into the acellular matrix implant.

The acellular matrix implant sealed within the lesion also becomes a repository of exogenous growth factors that pass through the bottom protective biodegradable polymer barrier layer in response to joint loading and hydrostatic pressure to which the joint is subjected when undergoing a normal physical activity such as walking, running or biking. Consequently, in response to the hydrostatic pressure load, these factors become more concentrated within the defect site and chondrocytes released from adjacent areas of the surrounding extracellular matrix migrate into the lesion with ensuing chondrocyte proliferation and initiation of the de novo extracellular matrix synthesis within the lesion.

Moreover, the acellular matrix of the implant fills the defect with a material that has a reduced stiffness relative to normal articular cartilage and permits deformation of the adjacent native cartilage matrix edges thereby increasing level of shear stress further resulting in increased release of soluble mediators that indicate matrix remodeling and chondrocyte migration into the acellular matrix implant.

The presence of the acellular matrix implant sealed to the adjacent cartilage boundaries thus creates conditions by which matrix remodeling enzymes, namely matrix metalloproteinases, aggrecanases and cathepsins, become concentrated at the defect site and initiate enzymatic opening of the adjacent extracellular matrix so that chondrocytes may migrate into the acellular matrix implant, be deposited within its matrix, begin to divide and proliferate and secrete the new extracellular matrix, ultimately leading to formation of a normal healthy hyaline cartilage.

C. Types of Acellular Matrix Implant

The acellular matrix implant provides a structural support for migration, growth and two or three-dimensional propagation of chondrocytes in situ. Generally, the acellular matrix is biologically biocompatible, biodegradable, hydrophilic and preferably has a neutral charge.

Typically, the implant is a two or three-dimensional structural composition, or a composition able to be converted into such structure, containing a plurality of pores dividing the space into a fluidically connected interstitial network. In some embodiments the implant is a sponge-like structure, honeycomb-like lattice, sol-gel, gel or thermo-reversible gelation hydrogel.

Typically, the implant is prepared from a collagenous gel or gel solution containing Type I collagen, Type II collagen, Type IV collagen, gelatin, agarose, hyaluronin, cell-contracted collagens containing proteoglycans, glycosaminoglycans or glycoproteins, fibronectins, laminins, bioactive peptide growth factors, cytokines, elastins, fibrins, synthetic polymeric fibers made of poly-acids such as polylactic, polyglycotic or polyamino acids, caprolactones, polycaprolactones, polyamino acids, polypeptide gels, copolymers thereof and combinations thereof. Preferably, the implant matrix is a gel, sol-gel, a polymer of an aromatic organic acid, a caprolactone polymer or a polymeric thermo-reversible gel. Most preferably the implant matrix contains aqueous Type I collagen.

The acellular matrix implant may be of a type of sponge, scaffold or honeycomb sponge, scaffold or honeycomb-like lattice or it may be a gel, sol-gel or thermo-reversible gel composition or it may be a polymer of an aromatic organic acid or an absorbable caprolactone polymer.

The acellular matrix implant may be produced as two or three-dimensional entities having an approximate size of the lesion into which they are deposited. Their size and shape is determined by the shape and size of the defect.

a. Acellular Sponges or Sponge-like Implants

In general, any polymeric material can serve as the support matrix, provided it is biocompatible with tissue and possesses the required geometry. Polymers, natural or synthetic, which can be induced to undergo formation of fibers or coacervates, can be freeze-dried as aqueous dispersions to form sponges.

In addition to collagen, a wide range of polymers may be suitable for the fabrication of sponges, including agarose, hyaluronic acid, alginic acid, dextrans, polyHEMA, and poly-vinyl alcohol alone or in combination.

Typically, such sponges must be stabilized by cross-linking, such as, for example, ionizing radiation. Practical example includes preparation of freeze-dried sponges of poly-hydroxyethyl-methacrylate (pHEMA), optionally containing additional molecules, such as gelatin, advantageously entrapped within. Incorporation of agarose, hyaluronic acid, or other bio-active polymers can be used to modulate cellular responses. All these types of sponges can function advantageously as implant matrices for the purposes of the present invention.

The gel or gel solution used for preparation of the sponge or sponge-like implant is typically washed with water and subsequently freeze-dried or lyophilized to yield a sponge like matrix able to incorporate the migrating chondrocytes withing the matrix. The acellular matrix implant of the current invention acts like a porous sponge when infiltrated with the migrating chondrocytes wherein the cells are distributed within the sponge pores, providing a mesh-like support permitting the chondrocytes to migrate and settle there, begin to divide and proliferate and secrete materials for generation of new extracellular matrix and eventually for generation of hyaline cartilage contiguous with the existing healthy surrounding cartilage.

One important aspect of the sponge implant is the pore size of the sponge matrix. Sponges having different pore sizes permit faster or slower infiltration of the chondrocytes into said sponge, faster or slower growth and propagation of the cells and, ultimately, the higher or lower density of the cells in the implant. Such pore size may be adjusted by varying the pH of the gel solution, collagen concentration, lyophilization conditions, etc., during implant fabrication. Typically, the pore size of the sponge is from about 50 to about 500 μm, preferably the pore size is between 100 and 300 μm and most preferably about 200 μm.

The pore size of the acellular matrix implant will be selected depending on the recipient. In the young recipient where the metalloproteinases are present naturally and active, the pore size will be smaller as the activated chondrocytes will rapidly proliferate through the pores and secrete extracellular matrix. In older recipients, the pores will be bigger as the migrating chondrocytes will be sluggish and will need more time to settle in the pores and proliferate.

An exemplary acellular matrix implant made of collagen is seen in FIG. 2. FIG. 2A is an example image of acellular collagenous matrix implant of size 4 mm in diameter and of 1.5 mm in thickness. The seeding density of this implant is between 300,000-375,000 chondrocytes per 25 μl volume corresponding to about 12-15 millions cells/ml. The cell density following the implantation of the acellular matrix implant is, of course, dependent on the rapidity of the migration of chondrocytes from the surrounding native cartilage and on their ability to divide and rapidity of their multiplication, however, the collagenous matrix of the implant has a capacity to accommodate this range of migrating cells.

The acellular sponge may be prepared according to procedures described in Example 1, or by any other procedure, such as, for example, procedures described in the U.S. Pat. Nos. 6,022,744; 5,206,028; 5,656,492; 4,522,753 and 6,080,194 or in co-pending applications Ser. Nos: 10/625,822, 10/625,245 and 10/626,459, herein incorporated by reference.

b. Acellular Scaffold or Honeycomb Implants

One type of the implant of the invention is an acellular scaffold, honeycomb scaffold, honeycomb sponge or honeycomb-like lattice. All these implants contain a honeycomb-like lattice matrix providing a support structure for migrating and dividing chondrocytes. The honeycomb-like matrix is similar to that of the sponge described above but has that typical pattern of the honeycomb. Such honeycomb matrix provides a growth platform for the migrating chondrocytes and permits three-dimensional propagation of the migrated and divided chondrocytes thereby providing a structural support for formation of a new hyaline cartilage.

FIG. 2B is a side view scheme of a honeycomb structure of the acellular matrix showing a collagen sponge and collagen gel with pore (*) size of each column of about 200-400 μm.

The honeycomb-like matrix is fabricated from a polymerous compound, such as collagen, gelatin, Type I collagen, Type II collagen or any other polymer, as described above for the sponge, having a desirable properties. In the preferred embodiment, the honeycomb-like acellular matrix implant is prepared from a solution comprising Type I collagen.

The pores of the honeycomb-like implant are evenly distributed within said honeycomb matrix to form a structure able of taking in and evenly distributing the migrated chondrocytes.

One preferred type of the acellular matrix implant is Type-I collagen support matrix fabricated into a honeycomb-lattice, commercially available from Koken Company, Ltd., Tokyo, Japan, under the trade name Honeycomb Sponge.

Acellular matrix implant of the invention thus may be any suitable biodegradable structure, gel or solution, preferably containing collagen. For the purposes of convenience in implanting, such implant is typically a gel, preferably a sol-gel transitional solution which, at above room temperature, changes the state of the solution from a liquid sol to a solid gel. The most preferred such solution is the thermo-reversible gelation hydrogel or a thermo-reversible polymer gel as described below.

c. Sol-Gel Acellular Matrix Implant

Another type of the acellular matrix implant is the implant matrix fabricated from sol-gel materials wherein said sol-gel materials can be converted from sol to gel and vice versa by changing temperature. For these materials the sol-gel transition occurs on the opposite temperature cycle of agar and gelatin gels. Thus, in these materials the sol is converted to the solid gel at a higher temperature.

The sol-gel material is a material which is a viscous sol at temperatures below 15° C. and a solid gel at temperatures around and above 37° C. Typically, these materials change their form from sol to gel by transition at temperatures between about 15° C. and 37° C. and are in a transitional state at temperatures between 15° C. and 37°. However, by changing the hydrogel composition, the transition temperature of the sol-gel may be predetermined to be higher or lower than those given above. The most preferred materials are Type I collagen containing gels and a thermo-reversible gelation hydrogel (TRGH) which has a rapid gelation point.

In one embodiment, the sol-gel material is substantially composed of Type I collagen and, in the form of 99.9% pure pepsin-solubilized bovine dermal collagen dissolved in 0.012 N HCl, commercially available under the trade name VITROGEN® from Cohesion Corporation, Palo Alto, Calif. One important characteristic of this sol-gel is its ability to be cured by transition into a solid gel form wherein said gel cannot be mixed or poured or otherwise disturbed thereby forming a solid structure optionally containing other components supporting the chondrocytes activation and migration. Sterile collagen for tissue culture may be additionally obtained from other sources, such as, for example, Collaborative Biomedical, Bedford, Mass., and Gattefosse, SA, St. Priest, France.

Type I collagen sol-gel is generally suitable and preferred material for fabrication of an acellular sol-gel implant.

d. Thermo-Reversible Gelation Hydrogel Implants

Additionally, the acellular matrix implant may be prepared from thermo-reversible materials similar to sol-gel which materials, however, have much faster point of transition, without hysteresis, from sol to gel and vice versa.

The thermo-reversible property is important for implantation of the acellular matrix implant into the lesion cavity as it may be implanted into the lesion cavity in its sol state whereby filling said cavity with the sol wherein the sol forms itself according to the exact shape of the cavity leaving no empty space or being too large or too small, as the case may be, for a prefabricated sponge or a honeycomb lattice. Following the warming of the sol emplaced within the articular lesion cavity to the natural body temperature, the sol instantly transitions and becomes a solid gel providing a structural support for the migrating chondrocytes from the surrounding native cartilage.

One characteristic of the sol-gel is its ability to be cured or transitioned from a liquid into a solid form and vice versa. This property may be advantageously used for solidifying the liquid or liquefying the solid gel acellular matrix implant within the cartilage lesion as well as for delivery, storing or preservation purposes of said acellular matrix implant. Additionally, these properties of sol-gel also permit its use as a support matrix by changing its sol-gel transition by increasing or decreasing temperature in the lesion, or exposing the sol-gel to various chemical or physical conditions or ultraviolet radiation.

In one embodiment, the acellular matrix implant is a thermo-reversible gelation hydrogel or gel polymer kept stored and implanted at temperatures between 5° C. and 15° C. At that temperature, the hydrogel is at a liquid sol stage and permits easy emplacement into the lesion as the sol. Once the sol is emplaced within the lesion, the sol is naturally or artificially subjected to higher temperature of about 30° C. and 37° C. at which temperature the liquid sol solidifies into solid gel. The gelling time is from about several minutes to several hours, typically about 1 hour. In such an instance, the solidified gel may itself become and be used as an implant or this sol may be loaded into a separate support matrix, such as a sponge or scaffold honeycomb implant.

The primary characteristic of the thermo-reversible gelation hydrogel (TRGH) is that upon its degradation within the body it does not leave biologically deleterious material and that it does not absorb water at gel temperatures. TRGH has a very quick sol-gel transformation which requires no cure time and occurs simply as a function of temperature without hysteresis. The sol-gel transition temperature can be set at any temperature in the range from 5° C. to 70° C. by the molecular design of the thermo-reversible gelation polymer (TGP), a high molecular weight polymer, of which less than 5 wt % is enough for hydrogel formation.

The thermo-reversible gelation hydrogel (TRGH), should be compressively strong and stable at 37° C. and below until about 32° C., that is to about the temperature of the synovial capsule of the joint which is typically below 37° C., but should easily solubilize below 30-31° C. to be able to be conveniently changed to the sol within the lesion cavity. The compressive strength of the TRGH must be able to resist compression by the normal activity of the joint.

The typical TRGH is generally made of blocks of high molecular weight polymer comprising numerous hydrophobic domains cross-linked with hydrophilic polymer blocks. TRGH has a low osmotic pressure and is very stable as it is not dissolved in water when the temperature is maintained above the sol-gel transition temperature. Hydrophilic polymer blocks in the hydrogel prevent macroscopic phase separation and separation of water from hydrogel during gelation. These properties make it especially suitable for safe storing and extended shelf-life.

In this regard, the thermo-reversible hydrogel is an aqueous solution of thermo-reversible gelation polymer (TGP) which turns into hydrogel upon heating and liquefies upon cooling. TGP is a block copolymer composed of a temperature responsive polymer (TRP) block, such as poly(N-isopropylacrylamide) or polypropylene oxide and of hydrophilic polymer blocks such as polyethylene oxide.

Thermally reversible hydrogels consisting of co-polymers of polyethylene oxide and polypropylene oxide are available, for example, from BASF Wyandotte Chemical Corporation under the trade name of Pluronics.

In general, thermo-reversibility is due to the presence of hydrophobic and hydrophilic groups on the same polymer chain, such as in the case of collagen and copolymers of polyethylene oxide and polypropylene oxide. When the polymer solution is warmed, hydrophobic interactions cause chain association and gelation; when the polymer solution is cooled, the hydrophobic interaction disappears and the polymer chains are disassociated, leading to dissolution of the gel. Any suitably biocompatible polymer, natural or synthetic, with such characteristics will exhibit the same reversible gelling behavior.

e) Acellular Gel Implants

The acellular matrix implants of the invention may alternatively be prepared from various gel materials, such as suspending gels, not necessarily thermo-reversible, which are commercially available and may be suitable for use as acellular matrix implants as long as they are biodegradable.

One example of such gel is polyethylene glycol (PEG) and its derivatives, in which one PEG chain contains vinyl sulfone or acrylate end groups and the other PEG chain contains free thiol groups which covalently bond to form thio-ether linkages. If one or both partner PEG molecules are branched (three- or four-armed), the coupling results in a gel network. If the molecular weight of the PEG chains used for the implant preparation is between 500 and 10,000 Daltons along any linear chain segment, the network will be open and suitable for receiving migrating chondrocytes, swellable by interstitial water, and compatible with living chondrocytes.

The coupling reaction of PEG can be accomplished, for example, by preparing 5 to 20% (w/v) solutions of each PEG separately in aqueous buffers or cell culture media. Just prior to implantation, thiol, PEG and the acrylate or vinyl sulfone PEG are mixed and infused into the lesion. Gelation will begin spontaneously in 1 to 5 minutes. The rate of gelation can be modulated somewhat by the concentration of PEG reagent and by pH. The rate of coupling is faster at pH 7.8 than at pH 6.9. Thus, by modifying the pH of the PEG containing mixture, the gelation process may be controlled to be faster or slower, as desired by the surgeon. Such gels are, however, typically not degradable within the body unless the additional ester or labile linkages are incorporated into the chain. PEG reagents may be purchased from Shearwater Polymers, Huntsville, Ala., USA; or from SunBio, Korea.

In a second alternative, the gelling material may be alginate. Alginate solutions are gellable in the presence of calcium ions. This reaction has been employed for many years to suspend cells in gels or micro-capsules. A solution of alginate (1-2%; w/v) in culture media devoid of calcium or other divalent ions is mixed in a solution containing calcium chloride which will gel the alginate. Analogous reactions can be accomplished with other polymers which bear negatively charged carboxyl groups, such as hyaluronic acid. Viscous solutions of hyaluronic acid can be gelled by diffusion of ferric ions.

f. A Polymer of an Aromatic Organic Acid Matrix

The acellular implant may also conveniently be made of a polymer of an aromatic organic acid. Polymers of this type have typically a negative charge and are thus preferred for use as a bone-inducing composition carriers. However, these type of compounds may also be used and are suitable for use as cartilage acellular implants.

g. Absorbable Caprolactone Polymers

The acellular implant may also be conveniently made of absorbable caprolactone polymers. These polymers are typically crystalline, low melting, epsilon-caprolactone polymers as described, for example, in U.S. Pat. Nos. 6,197,320; 5,529,736; 6,485,749; 6,703,035 and 6,413,539, all hereby incorporated by reference in their entirety.

The caprolactone polymers may be additionally combined with comonomers, such as glycolide, glycolic acid or lactones, linked ionically or covalently to amine or ester chains.

D. Biodegradable Implant

The acellular matrix implant of the invention is a temporary structure intended to provide a support for the migrating, dividing, proliferating and extracellular matrix secreting chondrocytes released from the surrounding cartilage.

Consequently, the implant of the invention must be fully biodegradable. Whether it is a sponge, honeycomb lattice, sol-gel, gel, TRGH, a polymer of the aromatic organic acid or a caprolactone polymer, in time, the delivered implant is disintegrated or incorporated into the existing cartilage and is subsequently degraded leaving no undesirable debris behind.

Overall, any of the acellular matrix implants for cartilage defects described above is suitable for implantation into a cartilage lesion of any size and shape and provides a support for a structural rebuilding of the cartilage by migrating chondrocytes therein from the surrounding healthy host cartilage. The implantation of the implant of the invention results in the generation of normal healthy hyaline cartilage and in complete healing of the cartilage defect.

III. Osteochondral Defects and Treatment Thereof

Lesions of the articular cartilage are often accompanied by lesions of the underlying bone. Such defects are thus a composite of cartilage and underlying bone. These defects are herein cumulatively called osteochondral defects.

A. Method for Treatment of Osteochondral Defects

The osteochondral defects are caused by injury of the cartilage and bone. The cartilage and bone are histologically two different connective tissues, as described above. Consequently, it is not possible to effectively treat both using the same methods and means and such treatment is thus complex and more difficult than a treatment of the cartilage lesion or chipped bone alone.

In one attempt to treat these complex injuries, a mosaicplasty technique was developed. The mosaicplasty, as already mentioned above, involves a removal of grafts from the healthy tissue and plugging such grafts into both the bone and cartilage lesions. An obvious defect of this technique is that in order to treat the injured site, surgeon has to remove, during the open surgical procedure, a healthy tissue from another site thereby disrupting the healthy tissue in the process.

When, however, the method of the current invention is used to treat these complex osteochondral injuries, it is possible to treat both the bone and cartilage lesions during the same surgery without need to remove and disturb the healthy tissue and/or undergo multiple surgeries required, for example, for allograft transplantation and other techniques.

The current method permits such dual treatment simultaneously by implantation of, in combination, an acellular matrix implant and a bone-inducing composition or a carrier comprising said composition comprising a bone-inducing agents further, preferably, in combination with one or two protective biodegradable polymer barriers.

In practice, during the same surgery, the surgeon first debrides both lesions and deposits the bone-inducing composition or a carrier comprising said composition into the bone lesion and covers said bone lesions with one or several layers of a protective non-toxic biodegradable polymer, preferably a modified highly polymerizable protective biodegradable polymer having the extended polymerization time no shorter than 2 minutes and no longer than 10 minutes, as described below in section IV. After the protective biodegradable polymer barrier polymerizes, typically within several minutes, preferably between 3 and 5 minutes, the acellular matrix implant is deposited into the cartilage lesion and covered with yet another layer of the protective biodegradable polymer barrier, herein called the top protective biodegradable polymer barrier.

In this way, the bone-inducing composition or a carrier comprising said composition is sequestered within the bone lesion and the bone forming agents, such as, for example, demineralized bone powder, calcium phosphates, calcium citrate, hydroxyapatite, organoapatite, titanium oxide, polyacrylate, alone or in combination, and a bone morphogenic protein and/or other known bone-inducing agents, such as growth factors or TGFs, for example, act as inducement for osteoblast migration from the surrounding bone without interference from the acellular matrix implant. As a consequence of this separation of the bone and cartilage lesions, there is no invasion of the hyaline cartilage into or formation of fibrocartilage in the bone lesion.

Conversely, when the acellular implant is separated from the bone-inducing composition or a carrier comprising said composition, there is no interference from any of the bone-inducing agents with the chondrocyte migration, extracelular matrix formation and generation of the hyaline cartilage. Each the bone and the cartilage are treated separately and yet simultaneously during one arthroscopic surgery.

The protective biodegradable polymer may be deposited over the bone-inducing composition or in some instances also under the bone-inducing composition, preferably as is, that is without any additional agents being added, or if desirable, it may be added to the bone-inducing composition or a carrier comprising said composition.

The bone-inducing composition or a carrier comprising said composition deposited within the bone defect covered with the first layer of the protective biodegradable polymer is left in the lesion in order to achieve the bone reconstruction and growth. Both the composition and the protective biodegradable polymer are aiding in a bone natural healing.

The acellular matrix implant implanted within the cartilage defect separated from the bone lesion by the first layer of the protective biodegradable polymer and covered with the top protective biodegradable polymer barrier is left in the cartilage lesion until it biodegrades when the hyaline cartilage replacement is formed.

Typical process for osteochondral defects repair is the cleaning and debriding the osteochondral defect, depositing the bone-inducing composition or a carrier comprising said composition containing the bone-inducing agents, up to the upper limit of the lesion in subchondral bone, applying the layer of the non-toxic protective biodegradable polymer over the composition and letting the protective biodegradable polymer barrier to polymerize. The polymerization typically happens within 3 to 5 minutes but if needed, it could be faster or slower, typically in from about 2 to about 10 minutes, depending on the polymer modification. After the protective biodegradable polymer barrier polymerizes, the surgery proceeds with implanting the acellular matrix implant into the cartilage lesion, as described above. The cartilage lesion containing the implant is then covered with a second layer of the non-toxic protective biodegradable polymer to seal and protect the lesion from the exterior.

The above described procedure is particularly suitable for treatment of osteochondral injuries as it permits dual treatment under different conditions being implemented during the same surgery.

One specific case of osteochondral defects is osteochodritis dissecans, where a focal lesion of the bone and cartilage results in a loose or totally dislocated osteochondral fragment. Currently the only available treatment requires three independent surgeries including biopsy harvesting of periosteum (first surgery), culturing cells, removal of the loose fragment (second surgery), introduction of the cultured cells into the lesion and bone-grafting (third surgery).

The current method, as described above, or modified to include a step of the fragment removal, during a single surgery, eliminates a need for two or three surgeries, as all steps necessary for repair of the osteochondritis dissecans are performed at the same time during one surgery.

B. Bone-Inducing Agents

Bone-inducing agents are compounds or proteins having a definite ability to promote formation of the bone.

The most suitable bone forming agents are demineralized bone powder (DMP), calcium phosphate, calcium citrate, hydroxyapatite, organoapatite, titanium oxide and growth factors, namely a group of growth factors known as bone morphogenic proteins (BMP), fibroblast growth factor (FGF), platelet derived growth factor (PDFG), epithelial growth factor (EGF), glioma derived factor (GDF) and transforming growth factor beta-1 (TGF-β1), intact or recombinant. These growth factors may be used individually and/or in combination with each other or with other bone-inducing factors.

Bone morphogenic proteins are typically identified by the abbreviation BMP and are further distinguished from each other by numbering, such as BMP-2, BMP-3, BMP-4, BMP-5, BMP-6, BMP-7, BMP-8 and BMP-14. Some of them are further identified by a generic name, such as, for example, BMP-3 is called osteogenin, BMP-3B is GDF-10, etc. The bone morphogenic proteins are administered generally in concentration (per carrier volume or weight) of from about 0.01 to about 5 mg/cm³, preferably from about 0.1 to about 1.5 mg/cm³ or from about 0.01 mg/g to about 5 mg/g, preferably from about 0.1 mg to about 2 mg/g.

The demineralized bone powder is particularly suitable to be used as a bone-inducing composition or as a bone-inducing carrier and no other compounds are needed to serve as bone inducer or supporting structure and necessary because the demineralized bone powder mimics microporous structure of the bone. Before depositing the DBP into the bone or subchondral bone lesion, the DBP may be conveniently dissolved in collagen or some other adhesive fluid or hydrogel which will permit its deposition into the lesion but itself will have no bone-inducing function.

C. Bone-Inducing Composition

The bone-inducing composition or a carrier comprising said composition of the invention comprises one or several bone-inducing agents listed above, in concentrations as disclosed. The bone-inducing composition may be administered as a powder, solution, gel, sol-gel, or hydrogel mixed in concentration given above or incorporated into a structure similar to that of the acellular implant, pre-prepared and implanted into the bone lesion or fracture. The composition prepared as TRGH, for example, is prepared as a sol solution and administered as such. The sol subsequently changes its state into the gel filling out the whole bone lesion. The bone-inducing agents may also be dissolved in PEG, collagen, alginate, etc., and deposited as such. It could also be soaked up in a second sponge system like the acellular matrix sponge described above.

The preferred mode for bone-inducing agents' deposition into the osteochondral or bone lesions is to dissolve the agent in an adhesive solute, such as diluted collagen, alginate and such like adhesives.

D. Bone-Inducing Carriers

A bone-inducing carrier or a carrier comprising bone-inducing composition is a carrier compound which is suitable for depositing said bone-inducing composition comprising at least one bone-inducing agent or, preferably, a combination of several agents into a bone lesion. Typically, the carrier will be a biodegradable porous matrix, hydrogel, sponge, honeycomb, a polymer of an aromatic organic acid, polycaprolactone or scaffold having large pores from about 50 to about 150 μm, which pores encourage migration of osteoblast. The carrier will also have an interconnecting small pores of about 0.1 to about 10 μm which connect the large pores, permit the osteoblast to settle within the carrier and provide a supporting matrix and connecting microstructure for supply of nutrient and other factors thereby permitting the bone formation. The surface of such carrier might be negatively charged encouraging pseudopod attachment of osteoblasts and their migration into the carrier resulting in the bone formation.

IV. Protective Biodegradable Polymers

Protective biodegradable polymers of the invention are non-toxic polymeric compositions suitable to form a barrier insulating and protecting the implant from the migration of cells, cell or tissue debris or other undesirable contaminants from the underlying bone or tissue or from the external environment. Herein, these polymer compositions are called the first, second, top or bottom biodegradable polymers. These polymers may be the same or different for use as a bottom or top protective barrier.

Generally, the implant is implanted into the cartilage or bone lesion between at least two layers, of top and bottom of biologically acceptable adhesive protective biodegradable polymer barriers.

In practice, the first (bottom) layer of the protective biodegradable polymer barrier is introduced into the lesion and deposited at the bottom of the lesion. The function of the first protective biodegradable polymer barrier is to prevent entry and to block the migration of subchondral and synovial cells of the extraneous components, such as blood-borne agents, cell and cell debris, etc. Before the implant is deposited, such debris could interfere with the integration of the acellular matrix implant. The second function of the first protective biodegradable polymer barrier is to contain enzymes, hormones and other components which are naturally present in the lesion and which are needed for chondrocyte activation, migration, secretion of other agents and proliferation of newly formed extracellular matrix and hyaline cartilage.

After the bottom polymer barrier is deposited and polymerizes, the acellular matrix implant is implanted over the first protective biodegradable polymer barrier and the second (top) protective biodegradable polymer barrier is placed over the acellular matrix implant and let polymerize. The presence of both these polymer barriers in combination with the acellular matrix implant results in successful activation of chondrocytes, their migration and integration into the implant matrix and ultimately in a formation of a new joint hyaline cartilage.

A. The First Bottom Protective Biodegradable Polymer

In a method for treatment of cartilage lesions, the first (bottom) protective biodegradable polymer barrier forms an interface between the introduced implant and the native tissue, such as subchondral bone or cartilage. The first protective biodegradable polymer barrier, deposited at the bottom of the lesion, must be able to contain migrating chondrocytes within the lesion, to protect the implant from influx of undesirable agents and to prevent chondrocyte migration into the subchondral space. Additionally, the first biodegradable polymer barrier prevents the infiltration of blood vessels and undesirable cells and cell debris into the implant and it also prevents formation of fibrocartilage. Primarily, however, this protective biodegradable polymer must be non-toxic to cell or tissue at a site where it is deposited.

In a method for treatment of osteochondral defects, the first (bottom) protective biodegradable polymer barrier forms a barrier between the cartilage lesion and the bone lesion. Because the cartilage and osteochondral defects are defects occurring in two qualitatively different tissues they require different treatments. As described above, the bone lesion is treated with the bone-inducing composition or a carrier comprising said composition while the cartilage lesion is treated with the acellular matrix implant. Moreover, it is not desirable that the enzymes present in the cartilage lesion activating chondrocyte migration mix with the bone-inducing agents and growth factors needed for bone lesion repair. When there is no separation of one tissue from another, it can easily lead to, for example, fibrocartilage ingrowing into the bone area and, in such an instance, instead of bone being replaced with the bone, it is replaced with the inferior fibrocartilage. Consequently, for treatment of osteochondral defects, the bottom protective biodegradable polymer barrier is deposited over the bone lesion filled with the bone-inducing composition, or a carrier comprising said composition, separating the bone lesion from the cartilage lesion implanted with the acellular implant. In this way, each the acellular implant and the bone-inducing composition can work independently and without interference from the other.

B. The Second Top Protective Biodegradable Polymer

The second (top) protective biodegradable polymer barrier acts as a protector of the acellular matrix implant or the lesion cavity on the surface and is typically deposited over the lesion after the implant is deposited therein and in this way protects the integrity of the lesion cavity from any undesirable effects of the outside environment, such as invading cells or degradative agents and insulates the acellular matrix implant in place after its deposition therein.

The second protective biodegradable polymer barrier also acts as a protector of the acellular implant implanted within a cavity formed between the two protective biodegradable polymer barriers. In this way, the second protective biodegradable polymer barrier is deposited after the implant is deposited over the first protective biodegradable polymer barrier and sequesters the implant within the cavity.

The third function of the second protective biodegradable polymer barrier is as a baseline for the formation of a superficial cartilage layer.

Performed studies described below confirmed that when the second protective biodegradable polymer barrier was deposited over the cartilage lesion, a growth of the superficial cartilage layer occurred as an extension of the native superficial cartilage layer. This superficial cartilage layer was particularly well-developed when the lesion cavity was filled with the thermo-reversible gel or sol gel thereby leading to a premise that such gel might provide a substrate for the formation of such superficial cartilage layer.

The second protective biodegradable polymer barrier or the protective biodegradable polymer barrier used for the separation of the bone and cartilage lesions may or may not be the same as the first protective biodegradable polymer barrier and the first and second protective biodegradable polymers may be utilized as a barrier between the bone and cartilage lesions but the different protective biodegradable polymer barrier may also be used for this purpose.

The first and the second protective biodegradable polymer barrier is preferably a cross-linked polyethylene glycol, as described below, with methylated collagen, also known as CT3, modified to be non-toxic to the cells and to have extended polymerization time according to the invention.

C. Properties of the Protective Biodegradable Polymers

The biodegradable polymers suitable for practicing the invention must have certain properties. Both, the first bottom or second top protective biodegradable polymer barrier of the invention, must be non-toxic to the cells and tissue, must have controllable polymerization time within a specified time frame, must be biologically acceptable, bioresorbable and biodegradable by any acceptable metabolic pathway, or be incorporated into the newly formed hyaline cartilage tissue, must be easy to use and possess adhesive and cohesive properties. The latter two properties are especially important with respect to large lesions, trochlear groove or patellar lesions which are subject to high shear stress. The protective biodegradable polymer must further be non-toxic and biologically acceptable and compatible with tissue. The polymer must also be flexible, soft and non-rigid because the hard polymer could cause abrasion of or extrusion of the protective biodegradable polymer barrier from the tissue site. The polymer must not interfere with the formation of new cartilage, or promote the formation of other interfering or undesired tissue, such as bone or blood vessels or fibrocartilage.

The protective biodegradable polymer barrier must controllably polymerize from a flowable liquid or paste to a load-bearing polymer within 2 to 10 minutes, preferably within 3-5 minutes. It is critical that the protective biodegradable polymer does not polymerize too rapidly as it would cause problems with its placement during the surgery and with its even distribution over the lesion. This is particularly true with arthroscopy. Polymerization time less than 2 minutes is unacceptable, impractical and undesirable. Polymerization times longer then 10 minutes, on the other hand, are not compatible with surgical time constraints. Additionally, the overall mode of use should be relatively simple because complex and lengthy procedures will not be accepted by surgeons.

Adhesive bonding of certain strength is required to attach the protective biodegradable polymer to the tissue and to insulate such tissue. Minimal possessing peel strengths of the protective biodegradable polymer of the invention should be at least 3N/m and preferably 10 to 30 N/m. Additionally, the protective biodegradable polymer must itself be sufficiently strong so that it does not break or tear internally, i.e., it must possess sufficient cohesive strength. The cohesive strength is preferably a tensile strength in the range of 0.2 MPa, preferably 0.8 to 1.0 MPa. Alternatively, a lap shear of the polymer of the polymer bond strength should have values of at least 0.5 N/cm² and preferably 1 to 6 N/cm².

Protective biodegradable polymers modified according to the invention possess the required characteristics. In the un-cured, or liquid state, such biodegradable polymers typically consist of two freely flowable polymer chains which are not cross-linked together, but are neat liquids dissolved in water, physiologically compatible aqueous solvents or buffers.

Specific biodegradable polymers of the invention are three part compositions comprising three different compounds, namely a sulfhydryl group-containing compound, a sulhydryl reactive group containing compound and alkylated collagen. Typically, the collagen is alkylated, preferably methylated or ethylated, natural or recombinant, intact or denatured, a sulfhydryl group-containing compound is polyalkylene oxide and sulfhydryl reactive group-containing compound is polyalkylene oxide. Preferably, a sulfhydryl group-containing compound is derivatized polyethylene glycol, most preferably a tetra-thiol-derivatized polyethylene glycol, and preferably the sulhydryl reactive group containing compound is derivatized polyethylene glycol, most preferably tetra-succinimidyl-derivatized polyethylene glycol or tetra-maleimidyl-derivatized polyethylene glycol, or a mixture thereof derivatized with polyethylene glycol.

For purposes of this invention, in the polymer composition used as a biodegradable polymer barrier wherein the biodegradable polymer has an extended polymerization time, each compound is provided either in powder, paste or liquid form before its deposition into or over the lesion. Before use, all three components are mixed and dissolved in water or buffer at pH 3-4 or alternatively, each can be dissolved separately or two polyethylene glycols may be mixed and dissolved separately from the methylated collagen.

Typically, the composition contains about 10 mg of methylated collagen, 100 mg of tetra-thiol polyethylene glycol having molecular weight approximately 10,000 and 100 mg of tetra-succinimidyl polethylene glycol having the molecular weight approximately 10,000. In one embodiment, tetra-succinimidyl polethylene glycol having the molecular weight approximately 10,000 may be replaced with a mixture of said succinimidyl polethylene glycol with maleimidyl polyethylene glycol in a ratio of about 1:1. The composition, dissolved in water or other acqueous solvent or buffer is then adjusted up to a volume of 1 ml with a phosphate/carbonate buffer and the pH is further adjusted with appropriate amount of acid to pH giving a controllable predetermined polymerization time, typically to pH lower than pH 8, preferably to pH below 7.5 with appropriate amount of hydrochloric or another acid.

Immediately upon mixing these three components, a cross-linking and polymerization reactions begin. The polymerization time is closely dependent on the pH under which the polymerization is performed. When the polymer chains are four-armed on at least one component, the coupling reaction leads to the formation of a polymeric network which is infinite in molecular weight. The methylated collagen reinforces the polymers formed from 4-armed PEGs of 10,000 Daltons (2500 Daltons per chain segment).

At the time of use as a polymeric barrier, some type of action to trigger the polymerization is required. Such triggering action can be the mixing of two reactive partners, the addition of a reagent to raise the pH as in the case of preferred modified protective biodegradable polymer barrier, or the application of heat or light energy.

In general, a protective biodegradable polymer barrier useful for the purposes of this application has adhesive, or peel strengths at least 10 N/m and preferably 100 N/cm; it needs to have tensile strength in the range of 0.2 MPa to 3 MPa, but preferably 0.8 to 1.0 MPa. In so-called “lap shear” bonding tests, values of 0.5 up to 4-6 N/cm² are characteristic of strong biological adhesives. The tensile strength of this cured gel is about 0.3 MPa.

One preferable protective biodegradable polymer barrier for use in this invention contains 4-armed tetra-succinimidyl ester PEG and tetra-thiol derivatized PEG, plus methylated collagen, known as CT3, commercially available from Cohesion Inc., Palo Alto, Calif., modified to alter its rapid gelation time into a polymerization time required by the method of the invention. Other polymers which could be advantageously used, when modified according to Example 4, in practicing of this invention are described in U.S. Pat. Nos. 6,312,725B1 and 6,624,245B2 and in J. Biomed. Mater. Res., 58:545-555 (2001), J. Biomed. Mater. Res., 58:308-312 (2001) and The American Surgeon, 68:553-562 (2002), all hereby incorporated by reference.

Another preferable protective biodegradable polymer barrier for use in this invention contains 4-armed tetra-succinimidyl ester PEG and tetra-thiol derivatized PEG, further with a recombinant alkylated collagen, for example methylated or combined ethylated collagen, similar to a polymeric adhesive known as CT3, commercially available from Cohesion Inc., Palo Alto, Calif.

The biodegradable polymer of the invention is thus modified CT3 adhesive having altered its rapid gelation time of 5 to 60 seconds into a polymerization time of 2 to 10 minutes required by the method of the invention. Other polymers which could be advantageously used in practicing of this invention, when modified according to Example 4, and using a recombinant rather than intact bovine collagen, are described in U.S. Pat. Nos. 6,312,725B1 and 6,624,245B2 and in J. Biomed. Mater. Res., 58:545-555 (2001), J. Biomed. Mater. Res., 58:308-312 (2001) and The American Surgeon, 68:553-562 (2002), all hereby incorporated by reference.

Another preferable protective biodegradable polymer barrier for use in this invention is comprised of the alkylated, preferably methylated collagen which is prepared recombinantly and is in combinations of cross-linking PEGs.

The composition of the invention is fully biodegradable. Degradation occurs through hydrolytic cleavage of ester bonds present in the succinimidyl ester PEG, releasing the soluble PEG chains which are excreted.

D. Modification of the Polymerization Time

Polymerization time of the protective biodegradable polymer composition is very important criteria for practicing the current invention. Until now, all known adhesives, glues, fibrin glues and sealants had been designed, intentionally and purposely, to have a very rapid, typically 5-30 seconds, at maximum 60 seconds, polymerization or gelling time to meet requirements for tissue sealant and adhesives. A reason is that these sealants or glues are used for sealing of wounds, stoppage of bleeding, preventing adhesion of tissues, etc, where time is of essence and these procedures are required to be performed very rapidly.

The current invention, on the other hand, involves a deposition of the polymer barrier into a debrided cartilage or bone lesion during arthroscopic surgery. Under the operating room conditions it is impossible for surgeon to prepare the polymer composition and to deposit it within the lesion in 5-30 or even 60 seconds, a time in which all known sealants and glues either gel or polymerize. The surgery, such as for example knee joint arthroscopy, involves manipulation of arthroscopic instruments and equipment in a precise manner under very defined conditions. The deposition of the polymer barrier under these conditions must correspond to the surgical time constrains. Consequently, the polymer barrier must be sustained in the non-polymerized state for much longer period of time then 60 seconds. The shortest possible polymerization time, which is possible but not preferred, is at least 2 minutes, with the most preferred time being between 3 and 5 minutes, for the surgeon to deposit the liquid polymer into the lesion and assure that it is evenly distributed over the whole bottom or over the surface of the lesion and completely polymerized before the implant is introduced into the lesion (for the bottom polymer) or the surgery is ended (for the top polymer). Partial polymerization or uneven distribution of the polymer barrier over the bottom of the lesion or over the lesion would lead to seeping through of undesirable blood components, metabolites or cell debris, which would defeat the purpose of the barrier.

Additionally, known sealants, particularly CT3 sealant, when not buffered, have acidic pH of around pH 3.4 which is nonphysiological and detrimental to the cells and tissues. Such acidic pH has been found to be toxic to the cells and tissues within the cartilage lesion.

Consequently, none of the previously known and available sealants and glues are suitable for practicing the current invention without modification. First, these sealants have very short polymerization time which is unacceptable for the purposes of this invention. Second, they show a substantial cell toxicity at a site of the biodegradable polymer barrier deposition which defeats the purpose of this invention, namely to grow the new cartilage. Thus, if these sealants are to be used as bottom or top barriers for the purposes of the current invention, they need to be modified to biodegradable polymers having an extended polymerization time, physiologically acceptable pH and no substantial cell or tissue toxicity.

E. Process for Modification of Polymerization Time

Modification of the polymerization time and elimination of cell toxicity of the existing sealants and polymers can be achieved with a fine tuning of the pH, buffer mixture ratio and ionic strength of the polymeric mixture comprising derivatized polyethylene glycols and methylated collagen, known as CT3, dissolved in a buffer. The pH, ionic strength and buffer composition are thus very important criteria for precise control of the polymerization time and for control of cell toxicity.

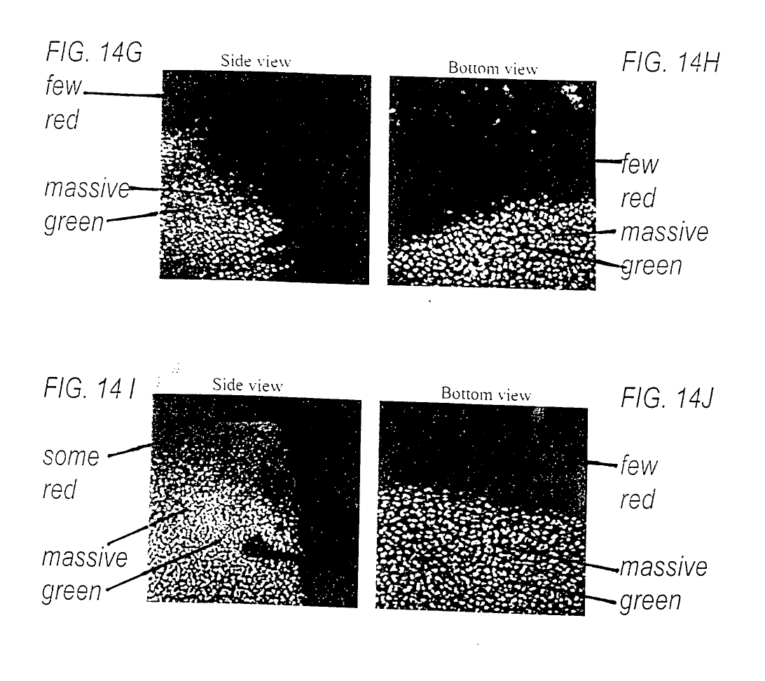

As already discussed above, the in situ applied unmodified sealants and glues, particularly CT3 sealant paste supplied commercially, have typically pH around 3.4. At such pH, when applied without buffer, CT3 is toxic to the cells and tissue, as seen in FIGS. 14C and 14D. When used as a tissue sealant without the current modification, CT3 is mixed, in a dual syringe air-spray system, with a phosphate/carbonate buffer at pH 9.6. In such a system, the mixture of the CT3 sealant with buffer having pH 9.6 results in a sealant having pH between 7.7 and 8.4 with a very rapid polymerization of between 5-10 seconds. Such fast polymerization is far too rapid to be useful for the purposes of this invention. Moreover, at this pH, a high degree of cell toxicity occurs. While such cell and tissue toxicity may not be an important factor for typical uses of the glues and sealants as tissue adhesives, it presents a major problem and becomes a critical issue for the current invention.

In the current invention, the success of the implanted acellular implant promoting generation of a new hyaline cartilage depends on migration of healthy cells from the surrounding healthy cartilage. If the polymer barrier has a toxic properties toward these cells at a site where the cells migrate into the acellular matrix, the cells would die. The death of the chondrocytes migrating into the lesion would defeat the purpose of this invention. The acellular implant is introduced into the lesion as a support matrix into which the activated living chondrocytes migrate, establish themselves within the implant and begin to produce extracellular matrix of the hyaline cartilage. If these cells would die, they would not be available to take residence in the implant and begin secrete a new extracellular matrix. Moreover, if these cells would die, they would be destroyed and produce the cell debris and metabolites which the invention is designed to prevent. The cell debris and metabolites could disturb the formation of the hyaline cartilage and lead to formation of fibrocartilage. The tissue toxicity thus becomes critical for the current use where the success of the acellular implant implantation depends on migration of healthy cells and the cell toxicity is undesirable and unacceptable as the produced cell debris would disturb the formation of the hyaline cartilage and lead to formation of fibrocartilage.

Consequently, a study was designed to investigate whether the polymerization time of the biodegradable polymer generally and CT3 particularly could be changed and extended into a controllable time period between 2 and 10 minutes without any cell toxicity.

a. Polymerization Time of CT3

One study involved investigation of effect of the CT3 buffer generally used for administration of the CT3 sealant. The first study was designed to investigate if the same CT3 buffer having different pH would extend a polymerization time of CT3.

The process was intended to modify the CT3 buffer pH to optimize it for slower CT3 polymerization time which would result in in situ polymerization of CT3 within 3-5 minutes with enough strength to provide adhesive properties required for the protective biodegradable polymer barrier of the invention.

The CT3 buffer recommended for use in admixture with the CT3 sealant has the composition shown in Table 1.

TABLE 1
CT3 Buffer (pH 9.6)

| | Concentration | Concentration |
|---|---|---|
| Component | (g/L) | (mM) |
| NaH₂PO₄H₂O | 17.25 | 125 |
| Na₂CO₃ | 21.1 | 199 |

When CT3 is provided as a paste or powder, it has pH 3.4. When it is dissolved in the CT3 buffer of pH 9.6, the resulting composition ha pH between 7.7 and 8.4 and polymerization time of between 5 and 10 seconds.

For this study, the pH of the CT3 buffer 9.6 was adjusted with hydrochloric acid to pH 8.5, pH 8 and pH 7.5 in an attempt to avoid tissue shock from CT3 fast polymerization under low pH (7.7-8.4) and permit a comfortable time for the polymer deposition conditions required for surgical procedure. Each buffer was mixed with the commercial CT3 in a ratio 1:1 (CT3:CT3 buffer) and then adjusted to a more acidic pH with hydrochloric acid. Neutralization capacity of the acid, strength of polymerized CT3 and time needed for complete CT3 polymerization were determined. Results are summarized in Table 2 below.

TABLE 2

| | Neutralization | Strength of | |
|---|---|---|---|
| CT3 buffer | capacity of | polymerized | Time at CT3 |
| pH | acid | CT3* | polymerization |
| 8.5 | Good | Sufficient | Extremely rapid |
| | (pH 6 to 7) | | Polymerization |
| | | | (5 sec) |
| 8.0 | Poor | Sufficient | Very rapid |
| | (pH 5 to 6) | | polymerization |
| | | | (10 sec) |
| 7.5 | Poor | Sufficient | Rapid |
| | (pH 5) | | polymerization |
| | | | (30 sec) |

*Adhesive strength and polymerization time of each CT3/buffer composition was compared to the strength and polymerization time using CT3/CT3 buffer pH 9.6.

As seen from results seen in Table 2, unmodified CT3 buffer at the lower pH 7.5 did somewhat slow the polymerization process but such slowdown was not sufficient to allow polymerization time to occur at between 2 and 10 minutes. Under the conditions of this study, at pH 7.5, the polymerization time was still 30 seconds, too fast for the purposes of this invention.

It became clear that if the protective biodegradable polymer barrier should be using the CT3 polymer mixture, it has to be further modified with the buffer having different ionic strength to pH from about 6.5 to about 7.5, preferably from about pH 6.5 to about pH 7.0. At this pH, using the modified buffer, the CT3 containing biodegradable polymer of the invention polymerizes in time required to achieve a slow polymerization time longer than 2 minutes, preferably between 3 and 5 minutes. This, in turn gives the surgeon enough time to apply the polymer over the bottom of the lesion and permits even distribution of the polymer over the bottom of the lesion during surgery before the polymerization occurs.

Additionally, at the pH of the modified buffers, the cell toxicity observed with unmodified CT3 buffer/CT3 composition is eliminated or substantially reduced as seen in FIGS. 14E-14H.

b. Effect of Buffering System on Polymerization Time

The modification of the polymerization time as applied to this invention was achieved using the properties of a physiologically controlled tissue buffering system.

In the plasma of the body, hydrogen ion concentration is controlled by the use of three buffering systems, bicarbonate, phosphate and protein.

Buffers act through a pair of substances involved in a reversible reaction by which one substance yields a hydrogen ion and a second substance can bind a hydrogen ion depending on the resident environmental hydrogen ion concentration. These pairs consist of a weak acid and a conjugate base. The relationship between the ionization states of each component depends on the hydrogen ion concentration and is expressed by the Henderson-Hasselbalch equation. This relationship is as follows:
pH=pK+log [base conjugate]/[weak acid]
where [base conjugate] is the concentration of the base component and [weak acid] is the concentration of the weak acid. The effectiveness of the buffer system is dependent in part on the dissociation constant K of the weak acid, such that [H+][weak base]/[weak acid]=K.

In the plasma, the carbonic acid and bicarbonate pair and the hydrogen phosphate and dihydrogen phosphate pair are the primary inorganic buffers. The protein buffer system created by the large number of peptide amino acids that can yield or bind hydrogen ion is the third major system controlling pH in plasma. The effect of the protein buffer system, because of its location, might be minimal in this particular instance.

In the process of delayed polymerization described herein, the initial pH of the polymerizing system is lowered to pH 6 to prevent polymerization. The subsequent delay of polymerization then follows the process of physiological buffering of the reaction mixture as the interstitial fluid components enter in the tissue compartment. This is a slower process that follows diffusion that lead ultimately to the adjustment of pH in the system to the physiologically regulated level of pH 7.4 at which point polymerization can begin.

The composite buffer system used for mixture of the polymerizing agent, such as CT3, contains both the bicarbonate and the phosphate buffer systems providing a two phase buffer transition. First, the phosphate buffer system holds the mixture at or near pH 6.0 based on one of the three pKs of phosphate followed by a transition through to the bicarbonate buffering system of pH 6.1 until the overall buffering system reaches equilibrium at pH 7.4.

c. Modification of CT3 Buffer

The CT3 buffer modification process involved pH adjustment of the CT3 with a buffer which generated different ionic conditions and different and more physiologically acceptable pH of the biodegradable polymer. This process resulted in extension of the polymerization time to and beyond 120 seconds and in defining a different polymerization times which meet the invention requirement for the protective biodegradable polymer barrier non-toxicity, strength, adhesivity and polymerization time.

Determination of necessary modification of CT3 buffer in order to make CT3 sealant suitable for use as a protective biodegradable polymer barrier having extended polymerization time needed for performing the surgery was performed by testing several buffer solutions. Therefore, buffers of different strength were prepared as seen in Table 3.

TABLE 3
Buffer Solutions for Biodegradable Polymer

| | Concentration of | Concentration of |
|---|---|---|
| pH of CT3 | NaH₂PO₄ | Na₂CO₃ |
| buffer | (mM) | (mM) |
| 8.0 | 153 | 147 |
| 7.5 | 162 | 138 |
| 7.0 | 195 | 105 |
| 6.5 | 223 | 77 |

As seen from Table 3, each buffer differed from the other buffers in their ratio of the phosphate and carbonate component and in their pH. These buffers were then mixed with equal amount of CT3 sealant paste having pH 3.4 into a modified CT3 biodegradable polymer barrier and their pH and polymerization times were determined.

Polymerization time of the CT3 modified into a biodegradable polymer of the invention at various pH are seen in Table 4.

TABLE 4
Polymerization Time of Modified CT3 Biodegradable Polymer

| pH of buffer | Time of | pH of Modified CT3 |
|---|---|---|
| (P/C buffer) | Polymerization (sec) | Biodegradable Polymer |
| 9.6 | 5-10 | 7.7-8.4 |
| 8.0 | 15-30 | 6.0-6.4 |
| 7.5 | 30-60 | 6.4-6.7 |
| 7.0 | 90-120 | 6.7-7.0 |
| 6.5 | 180-210 | 7.0-7.4 |
| No buffer | 300 | 3.4-4.0 |

As seen in Table 4, unbuffered CT3 paste can be polymerized in approximately 5 minutes, however such a low pH between 3.4 and 4 leads to a substantial cell toxicity making the unmodified CT3 sealant unsuitable for use as a biodegradable polymer barrier. Using the above described buffers, the polymerization times could be extended to time periods useful for surgical procedures according to the invention. For example, when the buffer of the pH 7.0 was investigated, the polymerization time could be extended from 60 seconds observed at pH 7.5 to 90-120 seconds with further extending the polymerization time to 180-210 seconds with lowering pH to 7.0 and 6.5, respectively. At pH 6. 5, the actual pH of the modified CT3 sealant is between 7.0 and 7.4, which is physiologically acceptable pH.

From the results of the studies described above, it is clear that modified CT3 buffer when used in conjunction with the CT3 polymer for preparation of the biodegradable polymer barrier for implantation into a cartilage showed slow polymerization with non-toxicity and similar adhesiveness of using CT3 original buffer (pH: 9.6).

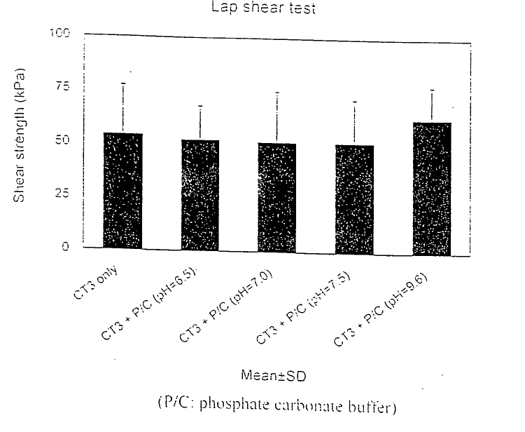

The adhesivity strength of the resulting modified CT3 polymer barrier measured by the lap sheer test is shown in FIG. 15. As seen in FIG. 15, the adhesivity strength of the pH modified CT3 did not change significantly.

V. Method for Formation of Superficial Cartilage Layer Over the Acellular Matrix Implant

An accompanying aspect of this invention is a finding that when the acellular matrix implant produced according to procedures described above is implanted into a cartilage lesion cavity and covered with a top protective biodegradable polymer barrier, the resulting combination allows the formation of a superficial cartilage layer completely overgrowing said lesion.

In practice, the method for formation of the superficial cartilage layer comprises several steps. First, the bottom of the lesion is covered with a first, bottom protective biodegradable polymer barrier deposited as slowly polymerizable solution. Following the polymerization of the protective biodegradable polymer barrier, the acellular matrix implant is implanted into said lesion and a second, top protective biodegradable polymer barrier is deposited over the implant and is let to polymerize. In one embodiment, the implant may be a thermo-reversible gel which easily changes from sol to gel at the body temperature thereby permitting an external preparation and delivery of the implant into the lesion. The gel is then covered with the top protective biodegradable polymer barrier which promotes generation of the superficial cartilage layer forming over the cartilage lesion thereby sequestering the implant within the lesion and protecting it from outside environment.

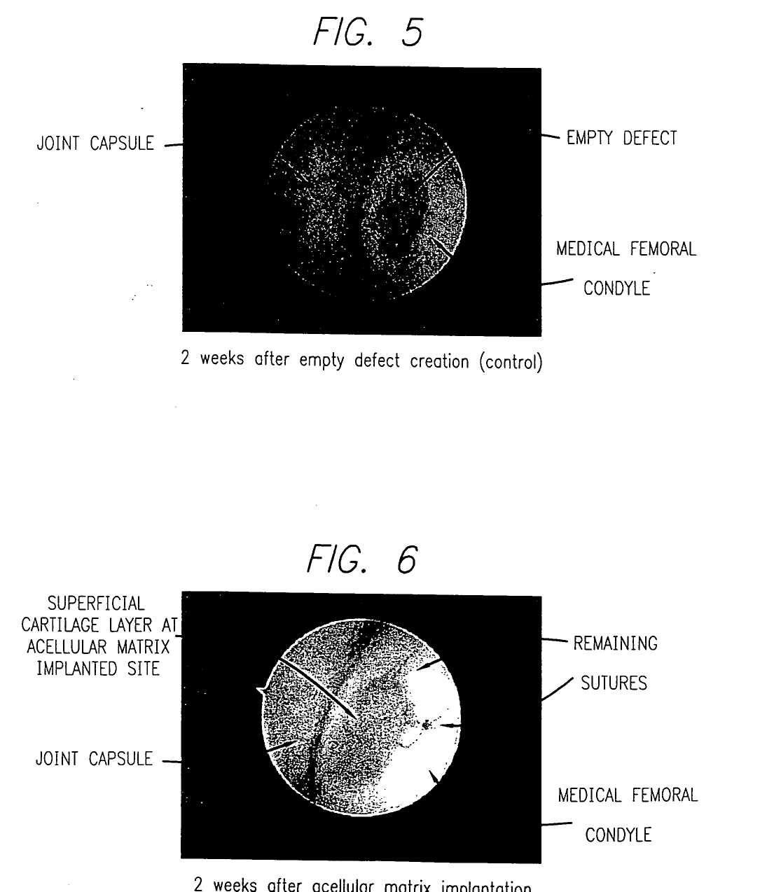

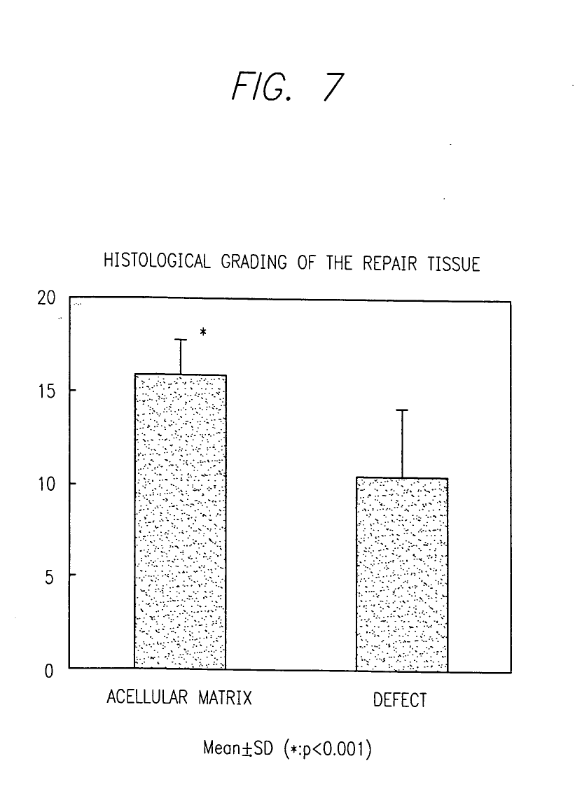

The superficial cartilage layer begins to form very quickly after the implant is implanted into the cartilage lesion and covered with the top protective biodegradable polymer barrier layer. As shown in FIG. 6, two weeks after acellular matrix implantation superficial cartilage layer was observed on the surface of acellular matrix implanted site. FIG. 6 shows arthroscopic evaluation two weeks after the defect was made in the femoral condyle where the superficial cartilage layer is clearly visible compared to untreated empty defect made at the same time, seen in FIG. 5.

The top protective biodegradable polymer barrier gives support and promotes formation of the superficial cartilage layer and, in some instances, is further assisted by the gel components of the matrix. At the time when the implant matrix is completely degraded and the new hyaline cartilage is formed in the defect, the superficial cartilage layer completely covers and insulates the newly formed cartilage similarly to a synovial membrane naturally present and covering the joints. The second top protective biodegradable polymer barrier is eventually also biodegraded and removed from the site, not however, until the superficial cartilage layer has formed.

VI. Method for Use of Acellular Matrix Implant

The method for repair and restoration of damaged, injured, diseased or aged cartilage to a functional cartilage is based on implantation of an acellular matrix implant into a cartilage lesion.

The method for use of the acellular matrix implant in these treatments comprises following steps:

a) Preparing an Acellular Matrix Implant

The first step involves preparation of the acellular matrix implant for implanting into the cartilage lesion. Preparation of acellular matrix implants is described in greater detail in sections II.A.

a) Selecting and Depositing the First and Second Protective Biodegradable Polymer Barrier into the Cartilage Lesion

The second step is optional but preferred and involves selection and depositing bottom and/or top protective biodegradable polymer barrier layers into a cartilage lesion.

Specifically, this step involves the preparation of a biodegradable polymer having a polymerization time between 2 and 10 minutes, preferably between 3 and 5 minutes, deposition of the first protective biodegradable polymer barrier at the bottom of the cartilage lesion and the second protective biodegradable polymer barrier over the acellular matrix implant. The first and the second protective biodegradable polymer barriers can be the same or different, however, both the first and the second protective biodegradable polymer barriers must have certain definite properties to fulfill their functions.

The bottom protective biodegradable polymer barrier, deposited into the lesion before the acellular matrix implant is introduced, acts as a protector of the lesion cavity integrity. It protects the lesion cavity from contamination by extraneous substances such as blood and tissue debris. It protects integrity of the naturally present enzymes and other mediators needed for and involved in formation of extracellular matrix and activation of chondrocytes and their migration from a surrounding host cartilage into the acellular implant implanted in the lesion. It also protects the lesion cavity from formation of the fibrocartilage.

The top protective biodegradable polymer deposited over the implant and effectively sealing the lesion from external environment acts as a protector of the lesion cavity, as a protector of the implant deposited within a lesion cavity formed between the two protective biodegradable polymer barriers and has sufficient biological permeability to permit the formation of the superficial cartilage layer.

c) Implanting the Acellular Matrix Implant

Next step in the method of the invention comprises implanting said acellular matrix implant into a lesion cavity formed between two layers of protective biodegradable polymer barriers.

The implant is preferably deposited into said lesion cavity after the bottom protective biodegradable polymer barrier is deposited but before the top protective biodegradable polymer barrier is deposited over it or the implant may be deposited into the lesion cavity without the bottom protective biodegradable polymer barrier being deposited there and then covered with the top protective biodegradable polymer barrier.

d) Generation of the Superficial Cartilage Layer

A combination of the acellular matrix implant comprising a matrix embedded with migrating chondrocytes with the top protective biodegradable polymer barrier leads to overgrowth and sealing of the lesion cavity with superficial cartilage layer.

Typically, a biologically acceptable top protective biodegradable polymer barrier, preferably a modified cross-linked PEG hydrogel with methylated collagen protective biodegradable polymer barrier (CT3), is deposited over the acellular matrix implant implanted into the lesion cavity. The second protective biodegradable polymer barrier acts as a baseline for formation of the superficial cartilage layer which in time completely overgrows the lesion and strongly resembles a healthy synovial membrane. In several weeks or months, usually in about two weeks, the superficial cartilage layer completely covers the lesion, protects the implant and migrating, dividing and proliferating chondrocytes and newly secreted extracellular matrix. Protecting the implant from extraneous environment permits integration of the newly formed cartilage tissue into the native surrounding cartilage substantially without formation of fibrocartilage.

Formation of the superficial cartilage layer is thus a very important aspect of the healing of the cartilage and its repair and regeneration.

VII. Method for Treatment of Cartilage Lesions

The method for treatment of damaged, injured, diseased or aged cartilage according to the invention is suitable for healing of cartilage lesions due to acute injury by providing conditions for regeneration of the healthy hyaline cartilage and for its integration into the surrounding native cartilage.

The method generally encompasses several novel features, namely, fabrication of a biologically acceptable biodegradable acellular matrix implant, selecting and depositing a top and bottom adhesive protective biodegradable polymer barriers to the lesion and the implantation of the acellular matrix implant within a cavity generated by two protective biodegradable polymer barriers, a formation of the superficial cartilage layer covering the lesion and protecting the integrity of the acellular matrix implant deposited therein, and providing conditions for activation, migration, dividing and proliferation of chondrocytes and for secretion of extracellular matrix ultimately leading to formation of the new hyaline cartilage and its integration into the native cartilage.

The method generally comprises steps:
a) fabrication of the acellular matrix implant according to the above described procedures;
b) debriding an articular cartilage lesion in surgical procedure;
c) during the debriding, preparing the lesion for implantation of the acellular matrix implant by depositing a bottom protective biodegradable polymer barrier at the bottom of the lesion thereby insulating said cavity from the surrounding tissue;
d) implanting the acellular matrix implant into said cavity formed by the polymerized bottom protective biodegradable polymer barrier to allow the activated and migrating chondrocytes to proliferate within said implant;
e) depositing a top protective biodegradable polymer barrier over the lesion, and thereby sealing said implant within the cavity formed between the two protective biodegradable polymer barrier layers;
f) optionally introducing enzymes, hormones, growth factors, proteins, peptides and other mediators into said sealed cavity by incorporating them into the acellular matrix, or coating said matrix with them, introducing them separately or generating conditions for their transport or transfer through the bottom protective biodegradable polymer barrier; and
g) following the surgery, subjecting an individual undergoing a surgery for repair of said lesion to a normal physical activity thereby naturally providing an intermittent hydrostatic pressure which was shown to promote formation of the healthy hyaline cartilage and its integration into the surrounding native intact cartilage.

There are several advantages of the current method.

The main advantage of this method is that the acellular matrix implant is prepared beforehand and is implanted during the first and only surgery where the cleaning and debriding is immediately followed by implantation of the acellular matrix implant.

Second, the acellular implant avoids immunological reactions to develop as there is/are no foreign tissue or cells involved because the implant is wholly synthetic and acellular. This is particularly true when the collagen in the biodegradable polymer is recombinantly prepared.

The method using the acellular matrix implant permits a three-dimensional expansion of chondrocytes and extracellular matrix.

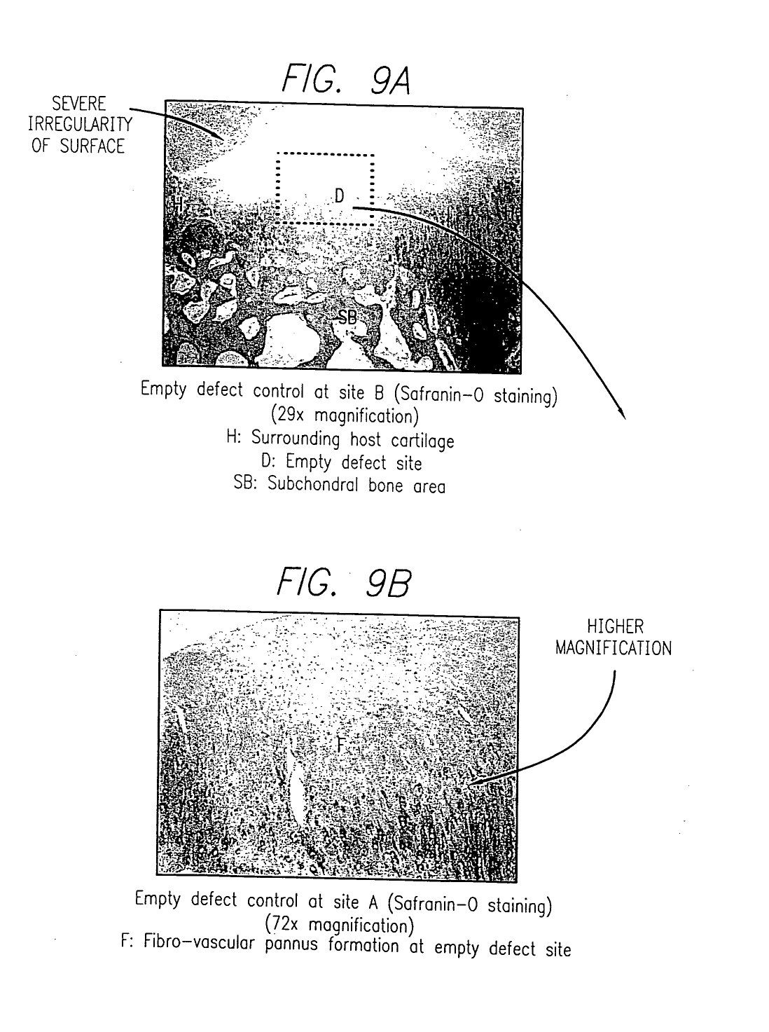

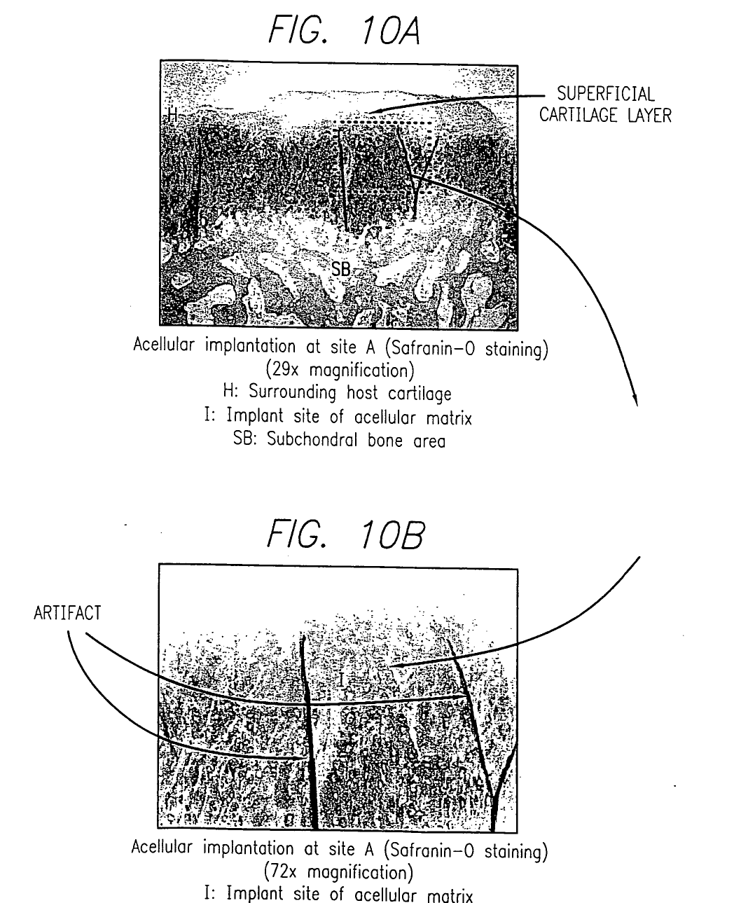

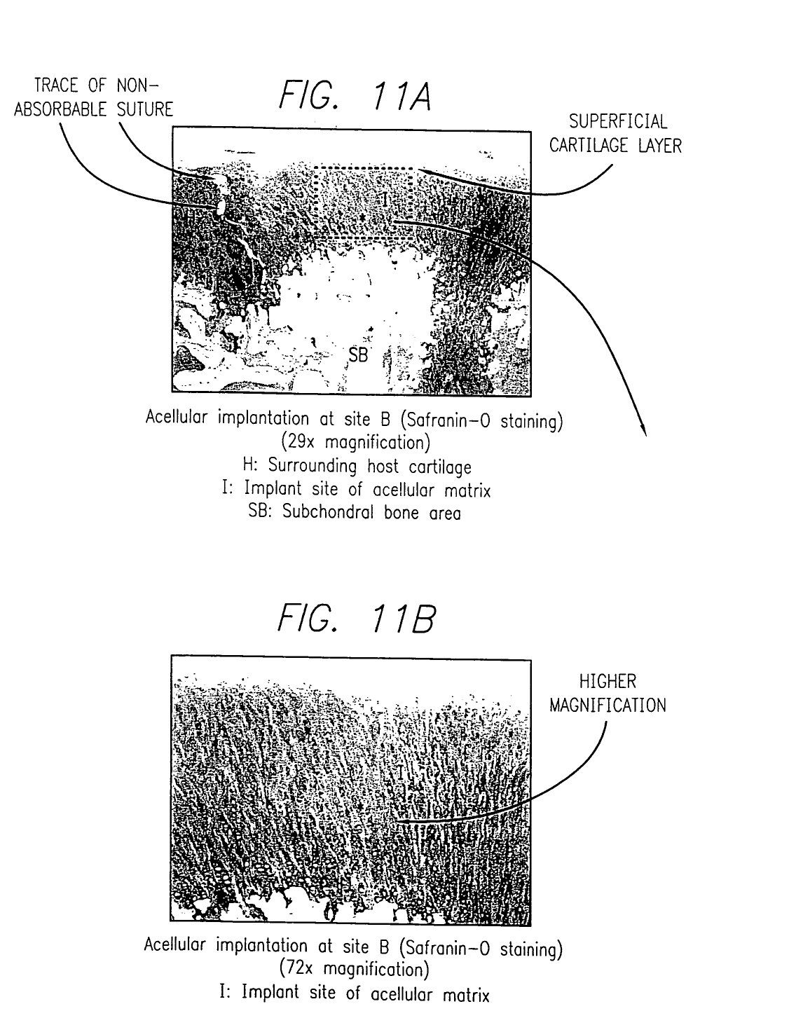

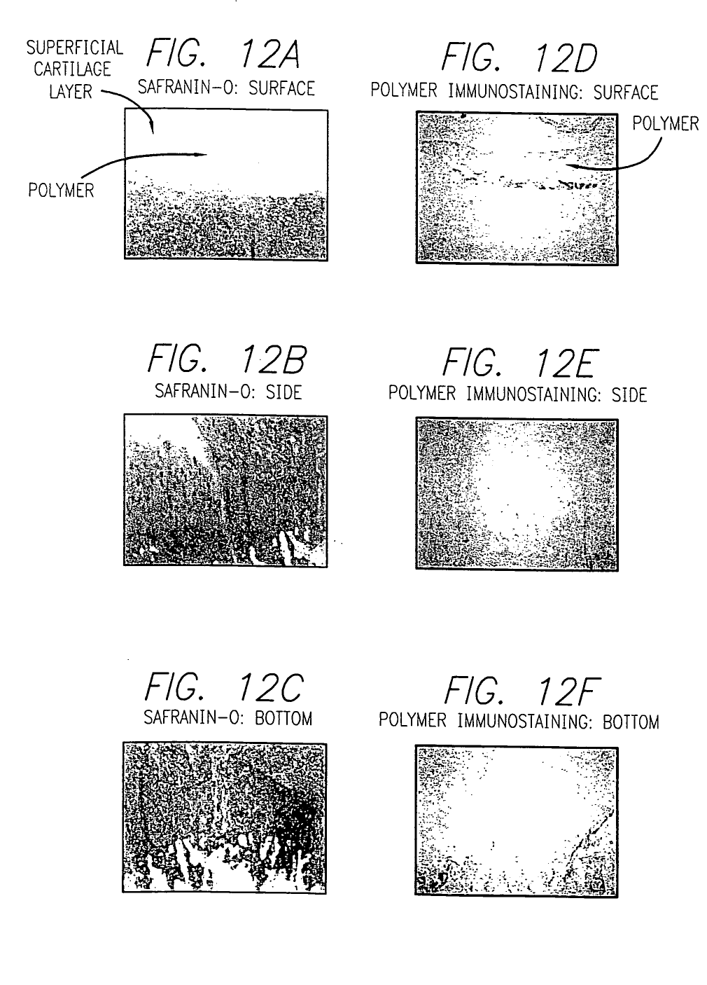

The deposition of the top protective biodegradable polymer barrier layer resulting in formation of superficial cartilage layer provides the outer surface of healthy articular cartilage overgrowing, protecting, containing and providing critical metabolic factors aiding in protecting the implant and activated migrating chondrocytes in the lesion. The superficial cartilage layer also prevent invasion of pannus (synovial membrane) into the lesion treated with the implant of the invention as seen in FIGS. 10A, 10B, 11A and 11B compared to untreated lesions seen in FIGS. 8A, 8B, 9A and 9B, where the presence of the invading pannus (synovial membrane) is clearly visible. In some instances, a selection of the thermo-reversible gel may be crucial as certain TRGH may function as a promoter for growth of the superficial cartilage layer without a need to apply the top protective biodegradable polymer barrier.

Deposition of the bottom protective biodegradable polymer barrier layer protects the integrity of the lesion after cleaning during surgery and prevents migration of subchondral and synovial cells and cell products thereby creating milieu for formation of healthy hyaline cartilage from the activated migrating chondrocytes into the acellular matrix implant and also preventing formation of the fibrocartilage.

The method further permits said acellular matrix implant to be enhanced with hyaluronic acid or other components or mediators named above, typically added in about 5 to about 50%, preferably about 20% (v/v), wherein such hyaluronic acid or such other components act as enhancers of the matrix-forming characteristics of the gel and also as a hydration factor in the synovial space in general and within the lesion cavity in particular.

Further, the method is very versatile and any of the implant type variations may be advantageously utilized for treatment of a specific cartilage, osteochondral or bone injury, damage, aging or disease.

For treatment of the cartilage, a subject is treated, according to this invention, with a prepared acellular matrix implant implanted into the lesion, the implant is left in the lesion coated with a bottom protective polymer barrier and covered with the top protective biodegradable polymer barrier for as long as needed. Usually, during the two-three months following the surgery and implant implantation, the new hyaline cartilage is formed and integrated into the native surrounding host cartilage. Typically also, there is no need for any further surgical or other intervention, as during these two-three months, at a normal physical activity, such as walking, running or biking, etc., a sufficient hydrostatic pressure is applied to the lesion to initiate and promote formation of the hyaline cartilage fully integrated into the native cartilage. Such cartilage will then become a fully functional cartilage covered with a superficial cartilage layer which eventually grows into or provides the same type of surface as a synovial membrane of the intact joint.

Finally, the method also permits replacement of the age worn out or diseased osteoarthritic cartilage by the regenerated hyaline-like cartilage when treated according to this invention.

The implantation protocol may assume any variation described above or possible within the realm of this invention. It is thus intended that every and all variations in the treatment protocol, the types of the implants, use of one or two protective biodegradable polymer barriers, all variations of the polymer barriers implantation process, selection of added mediators and not the least the normal physical activity of the individual are within the scope of the current invention.

VIII. Method for Treatment of Bone or Osteochondral Defects

The method for treatment of osteochondral defects is typically practiced in conjunction with treatment of cartilage. The method for treatment of bone defects and lesions may be practiced in conjunction with osteochondral defects or separately without steps involving deposition of the acellular implant into the cartilage.

A. Osteochondral Defects

Due to its anatomical arrangement where the subchondral bone is localized directly beneath the injured cartilage and the injury is both the injury to the cartilage and to the subchondral bone or subchondral skeletal bone, the method for treatment of osteochondral defects is an extension of the method for treatment of cartilage lesions described in section VII, with exception that during the step c) of that method, the surgeon, after debriding, deposits into the subchondral bone lesion a bone-inducing composition or a carrier comprising said composition typically comprising one or several bone-inducing agent(s), as described above, then covers said composition with a layer of the bottom protective biodegradable polymer barrier, and after permitting the protective biodegradable polymer barrier or the composition or both to polymerize, performs the steps a-g. This kind of osteochondral defect may further be such that the injury extends into the skeletal bone. In such an instance, the bone-inducing composition or bone acellular implant is deposited into the skeletal bone and in flowable continuation into the osteochondral bone which is then covered with the bottom protective biodegradable polymer barrier layer and the acellular implant is deposited as described above.

IX. Treatment of Human Osteoarthritic Cartilage

Articular cartilage is a unique tissue with no vascular, nerve, or lymphatic supply. The lack of vascular and lymphatic circulation may be one of the reasons why articular cartilage has such a poor intrinsic capacity to heal, except for formation of fibrous or fibrocartilaginous tissue. Unique mechanical functions of articular cartilage are never reestablished spontaneously after a significant injury, age wear or disease, such as osteoarthritis (OA).

Currently, the only available treatment of severe osteoarthritis of the knee is a total knee replacement in elderly patients. In young and middle aged patients, however, this is not an optimal solution.

Although the current invention is more practicable for treatment of injuries in young individuals who naturally possess sufficient levels of extracellular matrix building enzymes, growth factors, and other mediators, the method may be advantageously modified to also provide treatment for older population.

For treatment of elderly patients or for treatment of larger lesions, the acellular matrix implant is incorporated, before implantation, with one or more metalloproteinases, mediators, enzymes and proteins and/or with drugs stimulating endogenous production of these factors and mediators. These factors, as described above, stimulate and promote chondrocytes activation, migration and extracellular matrix secretion. The method of the invention thus is also suitable for treatment of the cartilage defects in older generation. It is expected, however, that such treatment will require longer period of treatment.

In osteoarthritis, or in age worn out cartilage, disruption of the structural integrity of the matrix by the degeneration of individual matrix proteins leads to reduced mechanical properties and impaired function. Consequently, the current invention reverses this process by providing a means for rebuilding the diseased osteoarthritic or worn cartilage with the new healthy hyaline cartilage.

X. In vivo Studies in Swine of the Weight-Bearing Region of the Knee

The method according to the invention was tested and confirmed in in vivo studies in swine.

The studies, described below, were designed to evaluate feasibility of porcine acellular matrix implant by detecting chondrocyte activation and migration into the surrounding cartilage, generation of newly synthesized hyaline cartilage within the lesion and formation of superficial cartilage layer.

Studies involved creation of defects in weight bearing region of femoral medial condyle of the knee joint, implantation of the acellular matrix sponge into the defect, depositing bottom and top protective biodegradable polymer barriers, detection of growth of a superficial cartilage layer after two weeks following the defect creation, detection of chondrocyte morphology, detection of pannus invasion and presence of fibrocartilage, detection of presence or absence of S-GAG secretion, histochemical evaluation of presence or absence of CT3 protective biodegradable polymer barriers.

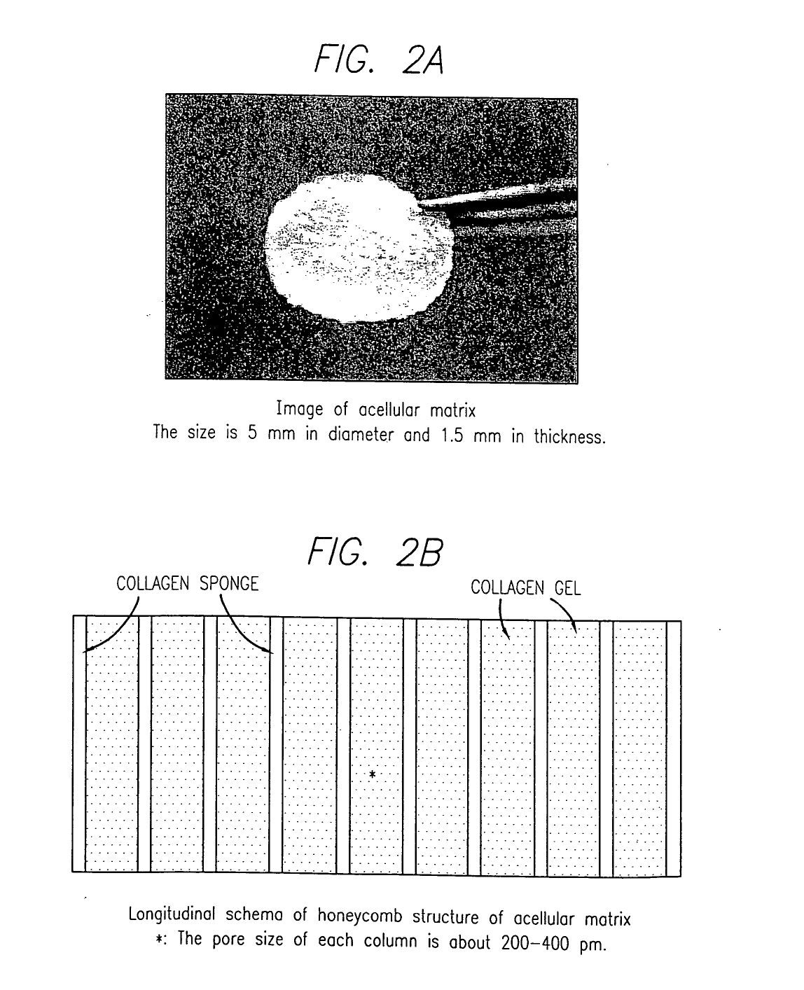

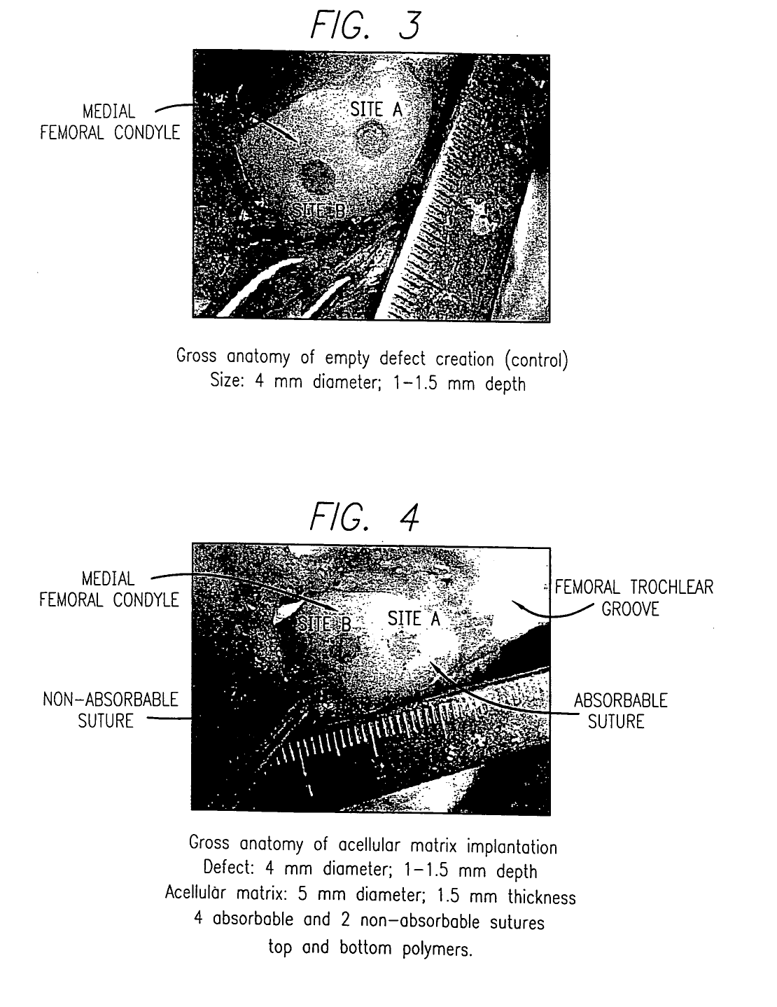

Gross anatomy of the empty defect creation and acellular matrix implantation at day zero is shown in FIGS. 3 and 4. Formation of the healthy hyaline cartilage and generation of the superficial cartilage layer in defects treated with the acellular matrix implant and the fibrocartilage pannus invasion in control defects at seven month following the defect creation are seen in FIGS. 5-12.

FIG. 3 shows two empty defects sites A and B at a time of the defect creation (time zero). FIG. 4 shows two defects created at time zero implanted with the acellular matrix implants at sites A and B.

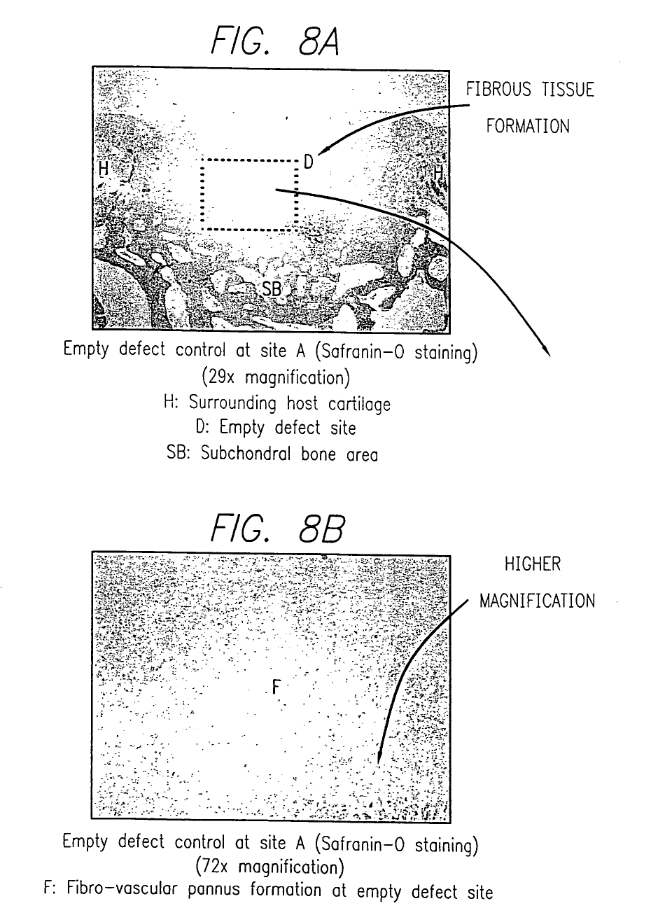

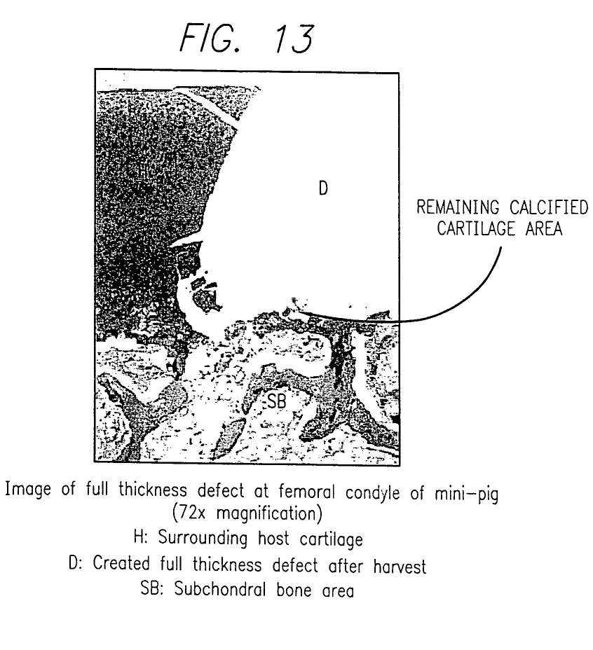

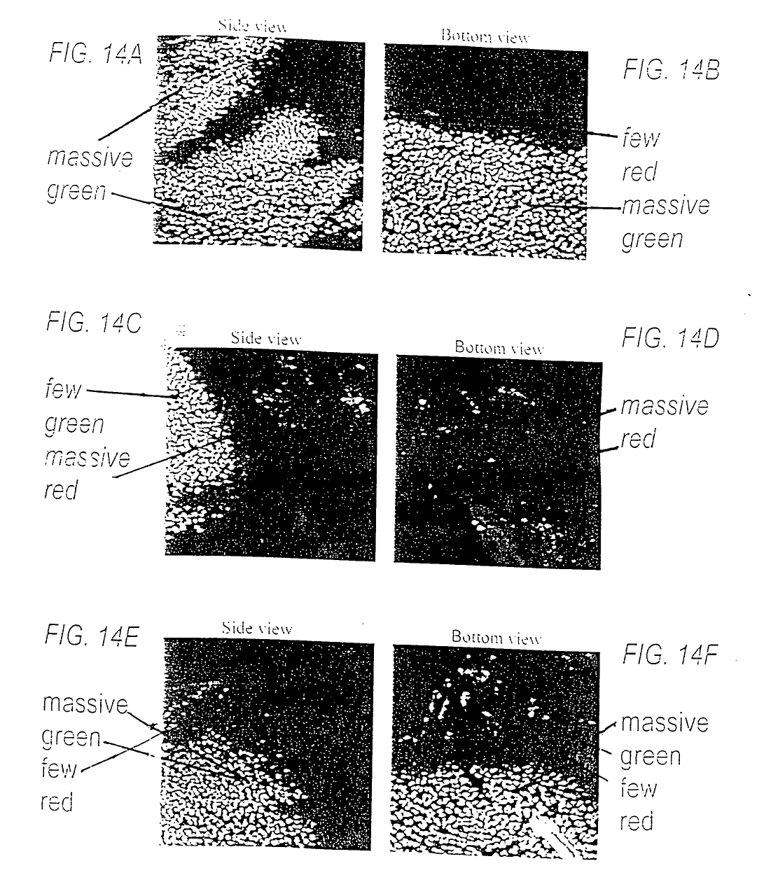

FIGS. 5 and 6 show arthroscopic evaluation two weeks after defect creation in the control (FIG. 5) and experimental animals (FIG. 6). Histological grading is seen in FIG. 7 and histological evaluation, in two magnifications, is seen in FIGS. 8 and 9 for the control animals and in FIGS. 10 and 11 for the experimental group treated with the acellular implant. Degradation of the bottom and top protective biodegradable polymer barriers from the cartilage lesion is seen in FIG. 12. One example of full thickness defect at femoral condyle of mini-pig is seen in FIG. 13. Cell toxicity of non-modified and modified CT3 sealant is shown in FIGS. 14A and 14H. A graph seen in FIG. 15 shows results of lap sheer test comparing CT3 modified with buffers of various strengths to different pH levels shown in FIGS. 14A-14H.

Schematic representation of the femoral articular surface, defect creation and implant implantation sites within said defect is shown in FIG. 2D. FIG. 2D shows two defects A and B created in the femoral medial condyle on the medial side of the femoral articular surface. The defects have sizes of 4 mm in diameter and 1.5 mm in depth. The defects are created in the weight-bearing region.

Table 5 is a tabulation of conditions of a study design as schematically illustrated in FIG. 2D.

TABLE 5
Study Design

| Group | Number of | Number of | | | |
|---|---|---|---|---|---|
| Number | Animals | Samples | Procedure | Arthroscopy | Necropsy |
| 1 | 8 | 16** | Implantation | 2 weeks after | 7 months after |
| Experimental | | | of acellular | implantation | implantation |
| | | | biodegradable | | |
| | | | matrix* | | |
| 2 | 8 | 16** | Empty defect | 2 weeks after | 7 months after |
| Control | | | control | defect creation | defect creation |

*Matrix was secured with tissue adhesive and sutures.
**Each group has two samples at weight-bearing site (site A and B, FIG. 2D).

Table 5 illustrates the study design for the seven months study of feasability of the acellular implant for treatment of cartilage lesions. Study involved 8 castrated male Yucatan micro-swine, 9-12 months old in each of the two groups. Two defects (A and B) were created at time zero in the knee of each animal, with a total number of 16 defects. The experimental group was implanted with acellular matrix implant at a time of defect creation. In the control group, the defect was left empty without any treatment and was used for visual, microscopical, histological and histochemical comparisons. Arthroscopy was performed at 2 weeks after implantation and defect creation. Necropsy was performed 7 months after implantation and defect creation.

The acellular matrix implant was prepared from a collagen solution VITROGEN® (35 μL) obtained from Cohesion, Calif. The collagen gel solution was absorbed into a collagen honeycomb sponge (5 mm in diameter and 1.5 mm in thickness) obtained from Koken Co., Japan. The combined collagen gel/sponge constructs (FIGS. 2A and 2B) were pre-incubated for 1 hour at 37° C. to gel the collagen, followed by incubation in culture medium with 1% penicillin and streptomycin at 37° C. at 5% CO₂. After about 24 hours of polymerization, the biodegradable scaffolds were transferred to the tissue container with pre-warmed culture medium (37° C.) for the implantation.

Arthrotomy was performed under an inhalation anesthesia. After opening knee joint capsule, two empty full-thickness defects (4 mm in diameter and about 1.5 mm in depth) were created in the medial articular cartilage on the weight-bearing site of the distal femoral condyle of each animal. After creating defects, tissue adhesive, typically modified cross-linked polyethylene glycol hydrogel with methylated collagen (CT3) protective biodegradable polymer barrier was placed on the bottom of the defect. Then, the preprepared acellular biodegradable sponge was placed over the bottom protective biodegradable polymer barrier within the cartilage lesion. The acellular sponge was secured with absorbable sutures (usually 4 to 6 sutures) and with two non-absorbable sutures. The non-absorbable sutures were used as a maker for gross observation and are visible in FIG. 6. The implanted defect was then sealed with the top protective biodegradable polymer barrier.

For the controls, two empty full-thickness defects were created and left intact, that is empty, without implants, or deposition of the bottom or top protective biodegradable polymer barriers.

FIG. 3 shows a photograph of the two empty full-thickness defects A and B (4 mm in diameter and 1-1.5 mm in depth) created in the medial articular cartilage on the weight-bearing site of the distal femoral condyle. The empty defects were left intact during the whole time of the study and were used as controls for the experimental group.

FIG. 4 is a photograph of the two full-thickness defects generated in the same way as the empty defects seen in FIG. 3. These two defects were treated, according to the method of the invention, with a bottom protective biodegradable polymer barrier deposited on the bottom of the lesion. The acellular implant was implanted into the lesion cavity over the bottom protective biodegradable polymer barrier and a top protective biodegradable polymer barrier deposited the over the implanted acellular matrix implant. The implants were collagenous sponges (FIG. 2A) and had 5 mm in diameter and 1.5 mm in thickness. Both sites A and B were implanted. Each implant was secured with four absorbable sutures and two non-absorbable sutures used as markers for future arthroscopic evaluation.

Two weeks after defect creation and acellular matrix implantation, the empty defects and implant sites were evaluated with arthroscopy. Arthroscopic evaluation after 2 weeks is seen in FIGS. 5 and 6.

FIG. 5 is an arthroscopic microphotograph of an empty defect 2 weeks after defect creation. Arthroscopic evaluation showed that in the control group, if left untreated, the lesion was invaded with synovial pannus and filled with fibrocartilage. The arthroscopic evaluation clearly shows the defect depression indicating that the defect is fully exposed and empty although some synovial invasion have already occurred. Such synovial invasion is a first step toward formation of fibrocartilage. Formation of fibrocartilage to replace the hyaline cartilage is undesirable as the fibrocartilage is qualitatively and functionally inferior to hyaline cartilage.

Arthroscopic evaluation of implanted sites showed that already at two weeks time the defects are covered with the artificial cartilage layer. FIG. 6 is an arthroscopic microphotograph of the defect treated with the acellular matrix implant 2 weeks after the defect was created. The FIG. 6 shows the superficial cartilage layer overgrowing the implant site forming a smooth flat surface. The borders of the implant site are already undefined compared to the empty defect which has a definite and visible border, said implanted site indicating the beginning of chondrocyte migration into the implant and secretion of extracellular matrix in confluency with the host cartilage, all this covered with the superficial cartilage layer. The arthroscopic evaluation seen in FIG. 6 revealed that the lesion implanted with the acellular matrix is unexposed and covered with the superficial cartilage layer completely overgrowing the implant sites, seen as a smooth flat surface when compared to the fully exposed and empty defects of controls, seen in FIG. 5.

At 7 months after creating the defects and implanting the acellular matrix implants, the animals were euthanized. The implant and defect sites on the femoral articular condyle were harvested for histological evaluation. The tissued were fixed with 4% formaldehyde/PBS for 7 days at 4° C. The tissues were decalcified with 10% formic acid, processed, and embedded in paraffin. Thin sections (5 μm) were stained with Safranin-O (Saf-O) and hematoxylin eosin (H-E) for histological evaluation.

The stained sections were evaluated blindly by means of a histological grading scale seen in FIG. 7, modified from J. Bone Joint Surg. Am., 79:1452-63 (1997). Only sections from the center of the defect were graded in order to ensure unbiased analysis and to allow comparison among specimens studied at different time-point. The area from the center of the defect was also chosen because it provided the most stringent test of healing capacity, since the least amount of cartilage healing was found consistently in specimens taken from the middle of the defect.

The histological scoring system used for cartilage repair evaluation is seen in Table 6.

TABLE 6
Category
1. Filling of defect

| Score | Filling of Defect |
|---|---|
| 0 | None (or almost none) |
| 1 | <50% |
| 2 | >50% |
| 3 | All (or almost all) |

2. Integration of repair tissue with surrounding articular cartilage

| Score | Integration |
|---|---|
| 0 | Gap or lack of continuity on two sides |
| 1 | Gap or lack of continuity on one sides |
| 2 | Non-continuous gap or lack |
| 3 | Normal continuity and integration |

3. Matrix staining with Safranin O-fast green
(compared to host cartilage)

| Score | Matrix staining |
|---|---|
| 0 | None (or almost none) |
| 1 | Slight |
| 2 | Moderate |
| 3 | All (or almost all) |

4. Cellular morphology

| Score | Chondrocytes morphology |
|---|---|
| 0 | Mostly spindle-shape (fibrous-like) cells |
| 1 | <50% of round cells with morphology of chondrocytes |
| 2 | >50% of round cells with morphology of |
| | chondrocytes |
| 3 | Normal (mostly round cells with morphology of |
| | chondrocytes) |

5. Architecture within entire defect (not including margins)

| Score | Architecture within entire defect |
|---|---|
| 0 | Clefts or fibrillations |
| 1 | <3 large voids |
| 2 | >3 large voids |
| 3 | Normal |

6. Architecture of surface

| Score | Architecture of surface |
|---|---|
| 0 | Severe fibrillation or irregularity |
| 1 | Moderate fibrillation or irregularity |
| 2 | Slight fibrillation or irregularity |
| 3 | Normal (or nearly normal) |

7. Penetration of tissue to subchondral bone area

| Score | Penetration |
|---|---|
| 0 | Severe penetration |
| 1 | Moderate penetration |
| 2 | Slight penetration |
| 3 | Normal (or nearly normal) |

Cumulative results of the histological grading of the repaired chondral cartilage is seen in Table 7.

TABLE 7
Histological Grading of the Repaired Cartilage

| | Acellular | |
|---|---|---|
| Category | Matrix Group | Empty Defect Group |
| Filling of defect | 3.00 | 2.60 |
| Integration | 2.00 | 1.40 |
| Matrix staining | 2.33 | 2.10 |
| Chondrocyte morphology | 1.78 | 0.80 |
| Architecture within entire | 2.33 | 0.30 |
| defect | | |
| Architecture of surface | 2.33 | 1.90 |
| Tissue penetration into | 2.11 | 1.40 |
| subchondral bone area | | |
| Average total score | 15.88 | 10.50 |
| SD± | 1.90 | 3.60 |

As seen in Table 7 the average total score for histological grading at 7 months after the defect creating and treatment with the acellular matrix implant was much higher in the implant group, with the score for all indicators in the implant group being higher then in the empty defect group.

Histological grading of the repair tissue is shown in FIG. 7, which graphically illustrates results shown in Table 5. The average total scores on the histological grading scale were significantly better (p≦0.001) for the defects treated with the acellular matrix implants than for the untreated defects.

At seven months following the defect creation, animals were sacrificed, their joints were harvested and evaluated by Safranin-O staining. Results are seen in FIGS. 8-11.

The non-implanted, empty defects A and B at 7 months after defect creating are shown in FIGS. 8A, 8B, 9A and 9B.

FIG. 8A is a Safranin-O staining microphotograph (29× magnification) of the empty, non-implanted defect D at a control site A seven months after defect creation. In higher magnification (FIG. 8B), the defect clearly shows a fibrous tissue (F) filling the defect surrounded by the host cartilage H with underlying subchondral bone (SB) area (FIG. 8A). None or a very small amount of S-GAG accumulation, depicted by red color, was observed at the defect site. S-GAG accumulation is evidence of the extracellular matrix formation. If there is a little or none S-GAG present, there is no extracellular matrix generated, indicating the absence of migrating chondrocytes and absence of formation of hyaline cartilage. It also indicates the presence and formation of fibrocartilage within the lesion.

FIG. 8B shows a 72× magnification of the defect site confirming a presence of fibroblasts, that is fibrous cells, indicating invasion of a fibrovascular pannus (F) from synovium. Chondrocyte morphology showed presence of mostly spindle (fibrous) cells.

FIG. 9A is a Safranin-O staining microphotograph (29× magnification) of the empty, non-implanted defect (D) at a site B of the control defect seven months after defect creation showing a formation of fibrous tissue filling the defect surrounded by the host cartilage (H) with underlying subchondral bone (SB) area. Severe irregularity of the lesion surface was observed. Only very slight S-GAG accumulation, depicted by red color, was observed at the defect site. S-GAG accumulation is evidence of the extracellular matrix formation.

FIG. 9B shows a 72× magnification of the defect site showing a presence of fibroblasts indicating a fibrovascular pannus F invasion from synovium. Cell morphology observed at this site shows mostly spindle fibrous cells.

FIGS. 8A, 8B, 9A and 9C clearly show that non-implanted control defects without treatment with the acellular implant of the invention do not indicate a formation of the healthy hyaline cartilage which would show as S-GAG accumulation, in Safranin-O stained microphotograps seen as a red color. Rather, these microphotographs show fibrovascular pannus synovial invasion into the defect with an accumulation of spindly fibrous cells present in the empty defect sites.

While the no-treatment of the lesion resulted in the filing of the defect with the fibrocartilage, the implantation of the acellular matrix implant into the defect induced chondrocyte activation and migration from the surrounding native cartilage and resulted in massive formation of cartilage extracellular matrix (ECM accumulation) with minimal fibrovascular pannus in the implant sites. ECM accumulation was detected by the strong red color present at the implanted sites of experimental animals. Results are seen in FIGS. 10A, 10B, 11A and 11B.

FIG. 10A is a micrograph of Safranin-O staining histological evaluation (29× magnification) of the acellular matrix implant (I) implanted withing the defect site A, seven month after defect creation and implantation of the acellular matrix implant. FIG. 10A clearly shows inducement of cell migration from the surrounding native host cartilage (H) into the implant (I) implanted within the defect site. After seven months following the implantation, hyaline-like cartilage was observed at the acellular implant site. The presence of the hyaline cartilage is indicated by the normal S-GAG accumulation, seen as a predominant red present in the defect site A. Superficial cartilage layer formed over the lesion is also seen. There was minimal fibrovascular pannus in the implant sites. Implant is surrounded by the host cartilage (H) with underlying subchondral bone area (SB).

FIG. 10B shows a higher magnification (72×) of the implant area with red color indicative of S-GAG accumulation and chondrocyte morphology showing primarily normal mostly round cells as compared to spindly fibrous cells observed in the non-treated control defects.

FIG. 11A is a Safranin-O staining histological evaluation (29× magnification) of the acellular matrix implant (I) implanted withing the defect site B, seven month after implantation. FIG. 11A confirms results seen in FIG. 10A. It clearly shows inducement of cell migration from the surrounding native host cartilage (H) into the implant (I) implanted within the defect site. At seven months after implantation, hyaline-like cartilage was observed at the acellular implant site. The presence of the hyaline cartilage was indicated by the normal S-GAG accumulation, seen as a predominant red color present in the defect site B. Superficial cartilage layer formed over the lesion and traces of non-absorbable suture are also seen. No fibrovascular pannus synovial invasion was observed in the implant site. Implant is surrounded by the host cartilage (9H) with underlying subchondral bone area SB. The non-absorbable suture indicates the original border between the host cartilage and the implant, now almost completely obscured.

FIG. 11B shows a higher magnification (72×) of the implant area with high accumulation of red color indicative of S-GAG presence. Chondrocyte morphology again show primarily normal, mostly round cells confirming results observed at site A.

As seen in FIGS. 10A and 10B, 11A and 11B, there was clearly visible integration between the biodegradable acellular matrix and the host cartilage. Such integration is not observed in FIGS. 8A and 9A where the defect is surrounded by the normal hyaline cartilage. These figures show different cell morphology at the defect sites than those at the implantation sites seen in FIGS. 10A and 10B. Cell morphology of the empty sites shows the presence of spindly fibrous cells dissimilar to those cells of the surrounding hyaline cartilage. Cell morphology at the implanted sites, on the other hand, show the presence of the normal (round) cells also observed in the surrounding healthy hyaline cartilage. The implanted site thus, after seven months does not show difference between the previously uninjured cartilage and the one formed within the defect following the implantation.

Additionally, the use of a top protective biodegradable polymer barrier deposited over the implant implanted at a defect site had resulted in formation of the superficial cartilage layer and minimizing synovial tissue invasion at the implant site.

A superficial cartilage layer is formed over the cartilage lesion after the top protective biodegradable polymer barrier is deposited over the lesion implanted with the acellular implant. As seen in FIG. 6, the presence of the superficial cartilage layer was already observed in two weeks after the implantation. The top protective biodegradable polymer barrier which causes the superficial cartilage layer to be formed is biodegradable and biodegrades within the time. At three months after the protective biodegradable polymer barrier deposition, remaining protective biodegradable polymer barrier was still observed at the surface area along with the superficial cartilage layer. At seven months after implantation, the top protective biodegradable polymer barrier was completely biodegraded and superficial cartilage layer was formed in its place, as seen in FIGS. 10A and 11A.

In order to determine the protective biodegradable polymer barrier (top and bottom) degradation in vivo, articular cartilage samples implanted with an autologous chondrocyte construct using the scaffold matrix were stained with Safranin-O (FIGS. 12A-12C) or treated immunohistochemically with monoclonal antibody for the protective biodegradable polymer barrier (modified CT3) and DAB (FIGS. 12D-12F). Reddish color in Safranin-O stained figures indicates S-GAG accumulation. Brown color indicates remaining protective biodegradable polymer barrier detected with diaminobenzidine (DAB).

FIG. 12 thus illustrates a degradation pattern, in time, of the top and bottom protective biodegradable polymer barriers three months after the acellular matrix implantation. At that time, the superficial cartilage layer was formed over the implant and the top protective biodegradable polymer barrier was partially degraded. The bottom protective biodegradable polymer barrier was, at three months following its deposition at the bottom of the lesion, completely degraded and removed from the lesion site.

FIG. 12A shows a surface view of the Safranin-O stained implantation site with the superficial cartilage layer clearly visible and the small amount of the top protective biodegradable polymer barrier (modified CT3) remaining under the superficial cartilage layer. FIG. 12B shows a side view of the Safranin-O stained implantation site. FIG. 12C shows the bottom view of the Safranin-O stained implantation site where at time zero the bottom protective biodegradable polymer barrier was deposited. FIG. 12D shows a surface view of the top protective biodegradable polymer barrier (modified CT3) immunostaining indicated by the brown color. FIG. 12E shows a side view of the top or bottom protective biodegradable polymer barrier immunostaining. FIG. 12F shows a bottom view of the bottom protective biodegradable polymer barrier (modified CT3) immunostaining.

In both tests, the remaining top protective biodegradable polymer barrier was observed only at the surface between the top of the regenerated hyaline like cartilage region and superficial cartilage layer (FIGS. 12A and 12D). There was no indication in side view of any remaining top or bottom protective biodegradable polymer barrier between the interface of the implant site and the surrounding host cartilage (FIGS. 12B and 12E). There was no remaining bottom protective biodegradable polymer barrier at the bottom of the lesion interfacing with the subchondral bone region where the bottom protective biodegradable polymer barrier was deposited at time zero (FIGS. 12C and 12F).

These results indicate that the bottom protective biodegradable polymer barrier is completely biodegraded and removed from the lesion site in about 3 month after implantation. At that time, there are still remnants of the top protective biodegradable polymer barrier visible on the surface of the lesion where the protective biodegradable polymer barrier protects the acellular implant from any migration or invasion of synovium and at the same time supports the formation of the superficial cartilage layer. With time even these remnants of the top protective biodegradable polymer barrier are biodegraded and removed from the healed lesion as evidenced by a complete absence of any top or bottom protective biodegradable polymer barrier at the defect site.

A reason why the top protective biodegradable polymer barrier in this case is still present at three months time is that, compared to the surface area, the side and bottom of the acellular implant site are more active regions for cell migration which is important for cell integration and formation of hyaline cartilage. In these regions, the protective biodegradable polymer barrier was completely degraded within 3 months. This phenomenon occurred and was observed in both the cellular and acellular matrix implantation in vivo. Cellular implant is described in copending application Ser. No. 10/625,245 filed on Jul. 22, 2003, herein incorporated by reference.

In order to confirm that the surgical technique used for creation of the cartilage defects in control and experimental animals is distinguished from the microfracture technique which penetrates the subchondral bone area, an image of full thickness defect at femoral condyle of mini-pig was created and is shown, with 72× magnification, in FIG. 13. FIG. 13 shows a paraffin embedded and Safranin-O stained reference tissue of the created full thickness defect. The defect was created of non-treated articular cartilage and bone from the femoral condyle surrounded by the host cartilage and underlying subchondral bone area. The remaining calcified cartilage area is seen in the area above the subchondral bone. This tissue was utilized in all studies as a reference tissue used for histological evaluation.

Safety of the modified biodegradable polymer barrier for the tissue was determined in all pH modified combinations of the CT3 sealant/buffer mixtures. As pointed out above, the undiluted CT3 sealant of pH 3.4 is toxic to the cells. Consequently, it was of importance to determine if the modified compositions would be also toxic or if they would avoid the cell toxicity observed with CT3 sealant. Study design for safety test included the implantation of the acellular matrix in porcine femoral condyle wherein the polymer barrier deposited at the bottom of the lesion was the unmodified CT3 sealant and the CT3 modified with buffers having a different pH. The implanted condyle was cultivated for 24 hours. The cultures were studied using a Live/Dead Staining Kit commercially available from Molecular Probes Inc, Oregon, USA. The implant sites were observed under confocal microscopy.

Live & Dead Cell Staining Kit provides a two-color fluorescence staining on live cells (green) and dead cells (red), using two probes. Calcein AM stains live cells green while Ethidium-III (ethidian homodimer-1) stains dead cells red. These probes are designed to measure two recognized parameters of cell viability.

High pH CT3 buffers (pH 8.0, 8.5, 9.0 and 9.6) were not measurable in this study due to their rapid polymerization of 5-10 seconds which makes them unsuitable for this invention.

Results are seen in FIG. 14. FIG. 14 illustrates toxicity of the unmodified sealant CT3 and CT3 sealant modified to a substantially non-toxic biodegradable polymer having an extended polymerization time, deposited into porcine femoral condyle, compared to untreated intact controls.

FIG. 14A (side view) and FIG. 14B (bottom view) show distribution of the living cells, stained green, and dead cells, stained red, from both views. Only very few dead cells were observed in non-treated group. Majority of the cells are stained green identifying them as living cells.

FIG. 14C (side view) and FIG. 14D (bottom view) show side and bottom view distribution of the living (green) and dead (red) cells observed after use of unmodified CT3 at pH 3.4 deposited as a bottom polymer barrier. In both side and bottom view the majority of dead cells is clearly visible. Only very few living cells are observed in side view and these may be attributed to the living cells of the surrounding tissue. This Figure clearly shows that unmodified CT3 is very toxic to the cells in the condyle lesion.

FIG. 14E (side view) and FIG. 14F (bottom view) show side and bottom view distribution of the living (green) and dead (red) cells observed after use of CT3 modified with a buffer to pH 6.5, used as a bottom polymer barrier. When the modified CT3 having pH of 6.5 was deposited into the condyle lesion, almost none of the cells observed were dead clearly showing that the modified CT3 biodegradable polymer barrier was not toxic cells and is eminently suitable for use in this invention.

FIG. 14G (side view) and FIG. 14H (bottom view) show side and bottom view distribution of the living (green) and dead (red) cells observed after use of CT3 modified with a buffer to pH 7.0, used as a bottom polymer barrier. When the modified CT3 having pH of 7.0 was deposited into the condyle lesion, majority of the cell observed were living cells with only a few cell being dead clearly showing that the modified CT3 biodegradable polymer barrier having a pH 7.0 was not toxic cells.

FIG. 14I (side view) and FIG. 14J (bottom view) show side and bottom view distribution of the living (green) and dead (red) cells observed after use of the CT3 modified with a buffer to pH 7.5, used as a bottom polymer barrier. When the modified CT3 having pH of 7.5 was deposited into the condyle lesion, more dead cells were observed with majority of the cells still being alive clearly showing even at the modified CT3 to pH 7.5 the biodegradable polymer barrier was not overly toxic cells.

FIG. 15 show results of the lap shear test in biodegradable polymers modified as shown in FIGS. 14A-14J. There was no significant difference of shear strength in all groups. Consequently, modifications of CT3 sealant did not in any way effect the adhesive strength of the biodegradable polymer used as a insulating barrier.

The results described above show that implantation of the biodegradable acellular matrix implant into the cartilage lesion according to the invention induces chondrocyte migration from surrounding native cartilage and formation of an extracellular matrix and leads to synthesis of a new hyaline cartilage with minimal synovial invasion of fibrovascular pannus at the implant sites.

Synthesis of the new hyaline cartilage was measured by the extracellular matrix accumulation expressed as accumulation of S-GAG. Also observed was a cell integration between the biodegradable acellular implant and the host cartilage. The use of a bottom and top protective biodegradable polymer barriers and sutures primarily to secure the implant within the defect suggest that these could have a secondary effect of minimizing synovial tissue invasion at the implant site. On the other hand, the results described above and illustrated by the figures clearly show that the intact nontreated control defects result in synovial invasion of the defect with fibrovascular pannus.

The acellular matrix implant most suitable for practicing the invention comprises a porous honeycomb sponge of type II atelocollagen filled with a thermoreversing hydrogel of Type II collagen sandwiched between a bottom layer and a top layer of a modified polyethylene glycol and methylated collagen (CT3) protective biodegradable polymer. The type I collagen cell walls of the porous honeycomb add further strength to the sealing capacity of the protective biodegradable polymer barrier by adding to the collagen-PEG chemical interaction analogously to the reaction of metal reinforcing bar to concrete.

The acellular implant itself is fully biodegradable in about 4 months. During that time the following conditions are observed in sexually mature but not fully epiphysealy-fused mini-swine. It is observed that in a 2 mm lesion of the femoral condyle covered with the top protective biodegradable polymer barrier, a superficial cartilage layer extending from the edge of the healthy cartilage region peripheral to the acellular implant proceeds to overgrow the lesion and the protective biodegradable polymer barrier layer. Additionally, chondrocyte migration into the acellular implant and production of the new hyaline cartilage matrix that eventually fills and replaces the implant is observed. This new cartilage matrix is or closely resembles hyaline cartilage as measured by sulfated glycoaminoglycan content and histological appearance. The source of these migrating chondrocytes are likely to be both the peripheral deeper layers of healthy chondrocytes peripheral to the acellular implant, and also the overgrown superficial cartilage layer, since it is shown that this layer is the source of differentiated chondrocytes capable of producing hyaline cartilage. Eventually hyaline-like cartilage is found to fill the implant while at the same time the implanted acellular matrix is gradually biodegraded.

In the current methodological arrangement the cells of subchondral origin cannot enter the implant. They are blocked by the bottom protective biodegradable polymer barrier facing the subchondral bone. Likewise, synovial pannus cannot enter the lesion because of the top protective biodegradable polymer barrier deposition. Only chondrocytes from the healthy host cartilage can enter the implant. Thus while the type I collagen alone can encourage cell migration, the protective biodegradable polymer barrier excludes migration except for healthy cartilage cells and possibly cartilage precursor cells from the surrounding healthy cartilage.

Furthermore, the hydrostatic pressure applied during the normal physical activity of the joint supports formation of true hyaline cartilage and contributes to the healing of the lesion.

Results of studies described above confirm that the damaged, injured, diseased or aged cartilage may be repaired by using acellular implants prepared according to the invention and that the acellular matrix implant of the invention induces cell migration from surrounding healthy host cartilage and its implantation induces the inward growth of the superficial cartilage membrane from the healthy tissue on the periphery. This membrane, superficial cartilage layer, protects the implant within the lesion from any synovial invasion. Once the implant is properly implanted within the lesion, the natural physicochemical factors, such as intermittent hydrostatic pressure, low oxygen tension and growth factors induce the cartilage recovery.

The advantages of the acellular matrix implant system are multiple. There is no need for biopsy and cell harvesting, no need to cover periosteum over the lesion, no damage to healthy tissue, the second and third surgery is eliminated resulting in faster recovery and elimination of waiting periods for the next surgery.

Advantages listed above are similarly attached to treatments of subchondral or bone lesions.

### EXAMPLE 1

### Preparation of Acellular Collagenous Implants

This example illustrates preparation of the acellular matrix implant.

300 grams of a 1% aqueous atelocollagen solution (VITROGEN®), maintained at pH 3.0, is poured into a 10×20 cm tray. This tray is then placed in a 5 liter container. A 50 ml open container containing 30 ml of a 3% aqueous ammonia solution is then placed next to the tray, in the 5 liter chamber, containing 300 grams of said 1% aqueous solution of atelocollagen. The 5 liter container containing the open trays of atelocollagen and ammonia is then sealed and left to stand at room temperature for 12 hours. During this period the ammonia gas, released from the open container of aqueous ammonia and confined within the sealed 5 liter container, is reacted with the aqueous atelocollagen resulting in gelling said aqueous solution of atelocollagen.

The collagenous gel is then washed with water overnight and, subsequently, freeze-dried to yield a sponge like matrix. This freeze dried matrix is then cut into squares, sterilized, and stored under a sterile wrap.

Alternatively, the support matrix may be prepared as follows.

A porous collagen matrix, having a thickness of about 4 mm to 10 mm, is hydrated using a humidity-controlled chamber, with a relative humidity of 80% at 25° C., for 60 minutes. The collagen material is compressed between two Teflon sheets to a thickness of less than 0.2 mm. The compressed material is then cross-linked in a solution of 0.5% formaldehyde, 1% sodium bicarbonate at pH 8 for 60 minutes. The cross-linked membrane is then rinsed thoroughly with water, and freeze-dried for about 48 hours. The dense collagen barrier has an inner implantation of densely packed fibers that are intertwined into a multi-layer structure.

In alternative, the integration layer is prepared from collagen-based dispersions or solutions that are air dried into sheet form. Drying is performed at temperatures ranging from approximately 4 to 40° C. for a period of time of about 7 to 48 hours.

For histological evaluation of the collagenous implant, 4% paraformaldehyde-fixed, paraffin sections were stained with Safranin-O (Saf-O) and Type II collagen antibody.

For biochemical analysis of the implant, seeded sponges were digested in papain at 60° C. for 18 hours and DNA content was measured using the Hoechst 33258 dye method. Sulfated glycosaminoglycan (S-GAG) accumulation was measured using a modified dimethylmethylene blue (DMB) microassay.

### EXAMPLE 2

### Biochemical and Histological Assays

This example describes assays used for biochemical and histological studies.

For biochemical (DMB) assay, the implant taken from the animal after certain time following the implantation, transferred to microcentrifuge tubes and digested in 300 μl of papain (125 μg/ml in 0.1 M sodium phosphate, 5 mM disodium EDTA, and 5 mM L-cysteine-HCl) for 18 hours at 60° C. S-GAG production in the implant is measured using a modified dimethylene blue (DMB) microassay with shark chondroitin sulfate as a control according to Connective Tissue Research, 9: 247-248 (1982).

DNA content is determined by Hoechst 33258 dye method according to Anal. Biochem., 174:168-176 (1988).

For histological assay, the remaining implants from each group were fixed in 4% paraformaldehyde. The implants were processed and embedded in paraffin. 10 μm sections were cut on a microtome and stained with Safranin-O (Saf O).

For immunohistochemistry, the samples are contacted with diaminobenzidine (DAB). The DAB is a color substrate showing brown color when the reaction is positive.

### EXAMPLE 3

### Evaluation of Integration of Acellular Matrix Implant in a Swine Model

This example describe the procedure and results of study performed for evaluation of integration of porcine in a swine model.

An open arthrotomy of the right knee joint was performed on all animals, and a biopsy of the cartilage was obtained.

A defect was created in the medial femoral condyle of the pig,s right knee. This defect (control) was not implanted with an acellular matrix implant but was left intact. Following surgery, the joint was immobilized with an external fixation implant for a period of about two weeks. Two weeks after the arthrotomy on the right knee was performed, an open arthrotomy was performed on the left knee and defects were created in this medial femoral condyle. The acellular matrix implant was implanted within the defect (s) in this knee which was similarly immobilized. The operated sites were subsequently viewed via arthroscopy two weeks after implantation or defect creation and thereafter at monthly intervals.

Animals were euthanized and the joints harvested and prepared for histological examination approximately 7 months after acellular matrix implant implantation. The implanted sites were prepared and examined histological.

### EXAMPLE 4

### Determination of CT3 Polymerization Time

This example describes a study used for determination of cell toxicity and polymerization time of CT3 sealant in attempts to be used as a protective biodegradable polymer barrier.

For its adhesive use, the CT3 sealant is provided as a paste or dry powder having pH 3.4. For the actual use it is mixed in two syringes air spray system with a CT3 buffer of pH 9.6. a composition of the buffer is shown in Table 1, above. The CT3 buffer contained a phosphate component (NaH₂PO₄ 17.25 g/L; 125 mM) and a carbonate component (Na₂CO₃ 21.1 g/L; 199 mM).

Three different CT3 buffers of pH 7.5, pH 8.0, and pH 8.5 were prepared using 1N hydrochloric acid solution for pH adjustment. The buffers pH is adjusted as seen in Table 2, above.

Target polymer barriers having pH 8.5, 8.0, 7.5 were mixed with CT3 in a mixture ratio 1:1 (CT3:CT3 buffer) and capacity of the neutralization acid, strength of the polymerized CT3 and the polymerization time at each pH were measured.

These parameters were compared to the polymerization time of about 5 seconds of the unmodified CT3 using the buffer pH 9.6.

The CT3 sealant using the above CT3 buffer has a polymerization time between 5 and 30 seconds even when the buffer is adjusted to pH lower that 9.6.

### EXAMPLE 5

### Determination of Polymerization Time of the Modified CT3

This example describes a process used for determination of necessary modification of CT3 sealant to be suitable for use as a protective biodegradable polymer barrier having extended polymerization time needed for performing the surgery.

The process is based on finding that the buffer pH, ionic strength and mixture ratio are very important for CT3 polymerization time.

Buffers of different strength were prepared as seen in Table 3, above.

Polymerization time of modified CT3 for use as a biodegradable polymer barrier and resulting pH of the modified CT3 were determined, as seen in Table 4, above.

1. A method for treatment of injury of an articulate cartilage and for repair and restoration of damaged, injured, diseased or aged cartilage to a functional cartilage, said method comprising steps:
a) preparing an acellular matrix implant;
b) coating a bottom of a cartilage lesion with a bottom protective biodegradable polymer barrier having a polymerization time at least 2 minutes; and
c) implanting said implant into said cartilage lesion.

2. The method of claim 1 wherein said bottom protective biodegradable polymer barrier deposited at the bottom of the lesion has a polymerization time between about 3 and 10 minutes.

3. The method of claim 2 wherein said bottom protective biodegradable polymer barrier has a polymerization time between about 3 and 5 minutes.

4. The method of claim 1 additionally comprising a step of depositing a layer of a top protective biodegradable polymer barrier over said implant implanted into said lesion.

5. The method of claim 4 wherein said top protective biodegradable polymer barrier deposited over said implant has a polymerization time of at least 2 minutes.

6. The method of claim 5 wherein said top protective biodegradable polymer barrier deposited over said implant has a polymerization time between about 3 and 10 minutes.

7. The method of claim 6 wherein said top protective biodegradable polymer barrier deposited over said implant has a polymerization time between about 3 and 5 minutes.

8. The method of claim 4 wherein said top and said bottom tissue protective biodegradable polymers are the same or different.

9. The method of claim 8 wherein said top and said bottom tissue protective biodegradable polymers are dissolved in a phosphate-carbonate buffer comprising from about 162 to about 223 mM of NaH₂PO₄ and from about 77 to about 138 mM of Na₂CO₃ having pH lower than pH 7.5 and the polymerization time between 3 and 5 minutes.

10. The method of claim 8 wherein said top and said bottom tissue protective biodegradable polymer are dissolved in a buffer having pH value between pH 6.5 and pH 7.0 and the polymerization time between 3 and 5 minutes.

11. The method of claim 10 wherein said tissue protective biodegradable polymer barrier comprises a combination of a modified polyethylene glycol and alkylated collagen.

12. The method of claim 11 wherein said polyethylene glycol is tetra-succinimidyl polyethylene glycol and tetra-thiol polyethylene glycol and wherein said collagen is methylated.

13. The method of claim 12 wherein said tissue protective biodegradable polymer barrier comprises methylated collagen combined with tetra-succinimidyl polyethylene glycol and tetra-thiol polyethylene glycol adjusted to pH lower than pH 7.0 said polymer having a polymerization time at least 3 minutes.

14. The method of claim 12 wherein said tissue protective biodegradable polymer barrier comprises about 10 mg of methylated collagen, about 100 mg of tetra-succinimidyl polyethylene glycol, about 100 mg of tetra-thiol polyethylene glycol per 1 mL of the phosphate-carbonate buffer adjusted to pH from about pH 6.5 to about pH 7.5.

15. The method of claim 13 wherein said phosphate/carbonate buffer comprises from about 195 mM to about 223 mM of NaH₂PO₄ and from about 77 mM to about 105 mM of Na₂CO₃ adjusted to pH from about 6.5 to pH of about 7.0.

16. The method of claim 11 wherein said tissue protective biodegradable polymer barrier is methylated collagen combined with tetra-functional sulfhydryl-polyethylene glycol and tetra-functional succinimidyl glutarate.

17. The method of claim 8 wherein said acellular matrix implant is a biodegradable collagenous sponge, honeycomb sponge, collagenous porous scaffold, gel, sol-gel, a polymer of an aromatic organic acid, caprolactone polymer or thermo-reversible gelation hydrogel (TRGH) matrix.

18. The method of claim 17 wherein the acellular matrix implant is prepared from a material selected from the group consisting of a Type I collagen, a Type II collagen, a Type IV collagen, a cell-contracted collagen containing proteoglycan, a cell-contracted collagen containing glycosaminoglycan, a cell-contracted collagen containing glycoprotein, a polymer of an aromatic organic acid, gelatin, agarose, hyaluronin, fibronectin, laminin, a bioactive peptide growth factor, a cytokine, elastin, fibrin, a synthetic polymeric fiber made of polylactic acid, a synthetic polymeric fiber made of polyglycolic acid, epsilon-caprolactone, a polyamino acid, a polypeptide gel, a polymeric thermo-reversible gelling hydrogel (TRGH), a copolymer thereof and a combination thereof.

19. The method of claim 18 further comprising a step of optionally introducing enzymes, hormones, growth factors, proteins, peptides and mediators, or drugs promoting an endogenous production of these factors or mediators, into said sealed cavity or generating conditions for their transport or transfer through the bottom protective biodegradable polymer barrier.

20. The method of claim 18 wherein said matrix additionally comprises matrix remodeling enzymes, matrix metalloproteinases, aggrecanases and cathepsins.

21. The method of claim 5 further comprising a step of subjecting an individual undergoing a surgery for repair of said lesion to a normal physical activity thereby providing an intermittent hydrostatic pressure.

22. The method of claim 21 suitable for treatment of the articular cartilage injury comprising steps:
a) preparation of an acellular matrix implant;
b) debriding an articulate lesion during the surgery;
c) preparation of a cartilage lesion for implantation of said implant, including a step of depositing a bottom protective biodegradable polymer barrier at the bottom of the cartilage lesion for separating and protecting said implant;
d) implanting the implant into the lesion;
e) depositing a top protective biodegradable polymer barrier over the acellular matrix implant; and
f) following the surgery, subjecting an individual undergoing a surgery for repair of said lesion to a normal physical activity thereby naturally providing an intermittent hydrostatic pressure.

23. A method for treatment of osteochondral defects, said method comprising steps:
a) preparing a bone-inducing composition or an implant carrier comprising said composition, said composition comprising one or several bone-inducing agents for implantation into a bone lesion;
b) preparing an acellular matrix implant for implantation into a cartilage lesion as a collagenous sponge, collagenous porous scaffold, a polymer of an aromatic organic acid, caprolactone polymer or thermo-reversible gelation hydrogel (TRGH) matrix support wherein said sponge, scaffold, polymer or TRGH are biodegradable, will disintegrate with time and be metabolically removed from the healed lesion and replaced with a hyaline cartilage, said matrix optionally comprising matrix remodeling enzymes, matrix metalloproteinases, aggrecanases and cathepsins;
c) introducing said bone-inducing composition or a carrier comprising said composition into a bone lesion;
d) covering said bone-inducing composition or a carrier comprising said composition with a bottom protective biodegradable polymer barrier;
e) implanting said acellular matrix implant into said cartilage lesion over the bottom protective biodegradable polymer barrier; and
f) introducing a layer of a top protective biodegradable polymer barrier over said implant wherein said top and bottom protective biodegradable polymer barriers may or may not be the same and wherein a combination of said acellular matrix implant and said top protective biodegradable polymer protects the implant deposited into the cartilage lesion.

24. The method of claim 16 wherein said inducing agent is selected from the group consisting of a demineralized bone powder, hydroxyapatite, organoapatite, calcium phosphate, titanium oxide, poly-L-lactic acid, polyglycolic acid, a copolymer thereof and a bone morphogenic protein.

25. The method of claim 17 wherein said bone-inducing agent is hydroxyapatite.

26. The method of claim 17 wherein said bone-inducing agent is the demineralized bone powder.

27. The method of claim 19 wherein said demineralized bone powder is dissolved in collagen.

28. The method of claims 16 wherein said carrier comprising the bone-inducing agent is a polymer of an aromatic organic acid.

29. A protective biodegradable polymer for top or bottom insulation of a cartilage or bone lesion wherein said protective biodegradable polymer is non-toxic to cells or tissue within the lesion, and is bioresorbable, biodegradable and biologically compatible with cartilage or bone tissue.

30. The protective biodegradable polymer of claim 29 wherein the protective biodegradable polymer barrier is a rapidly gelling polymer from a flowable liquid or paste to a load-bearing gel within 2 to 5 minutes.

31. The protective biodegradable polymer of claim 30 possessing a minimal peel strengths of at least about 3N/m to about to 30 N/m, a cohesive strength, measured as tensile strength in the range of from about 0.2 MPa to about 1.0 MPa or has a bond strength of at least 0.5 N/cm² to about 6 N/cm².

32. The protective biodegradable polymer of claim 31 wherein said protective biodegradable polymer is a gel having a cohesive strength dependent on the number of inter-chain linkages.

33. The protective biodegradable polymer of claim 32 wherein the pH of said protective biodegradable polymer is adjusted to or below pH 7.5.

34. The protective biodegradable polymer of claim 33 which has adhesive or peel strengths at least 10 N/m and tensile strength at least 0.3 MPa.

35. The protective biodegradable polymer of claim 33 which has adhesive or peel strengths of 100 N/cm and tensile strength in the range from 0.8 to 1.0 MPA.

36. The protective biodegradable polymer of claim 35 which is 4-armed polyethylene glycol derivatized with succinimidyl ester and thiol plus methylated collagen (CT3) in combination with a buffer having pH between 6.5 and 7.5.

37. The protective biodegradable polymer of claim 36 which is 4-armed tetra-succinimidyl ester or tetra-thiol derivatized PEG, plus methylated collagen.

38. The protective biodegradable polymer of claim 37 which is modified polyethylene glycol and methylated collagen to have a polymerization time between from about 3 to about 5 minutes.
